(19) United States
(12) Patent Application Publication
(10) Pub. No.: US 2022/0119538 A1
(43) Pub. Date: Apr. 21, 2022

(54) ANTI-IL-36R ANTIBODIES FOR TREATMENT OF CHRONIC INFLAMMATORY PAIN

(71) Applicant: Boehringer Ingelheim International GmbH, Ingelheim am Rhein (DE)

(72) Inventors: Christian THOMA, Biberach an der Riss (DE); Dirk ESSER, Kelkheim (DE); Tristan GLOEDE, Darien, CT (US)

(21) Appl. No.: 17/485,980

(22) Filed: Sep. 27, 2021

(30) Foreign Application Priority Data
Sep. 30, 2020 (EP) 20199185.8
Mar. 17, 2021 (EP) 21163078.5

(51) Int. Cl. C07K 16/28; A61P 29/00

(52) U.S. Cl. CPC C07K 16/2866; A61K 2039/545; A61P 29/00

(57) ABSTRACT

The present invention provides a method for treating or reducing chronic inflammatory pain in a subject by administering to the subject a dose of an anti-IL-36R antibody.

### SEQUENCE LISTING

The instant application contains a Sequence Listing which has been submitted in ASCII format via EFS-Web and is hereby incorporated by reference in its entirety. Said ASCII copy, created on Sep. 23, 2021, is named 09-0707-US-1-2021-09-27-SL.txt and is 147,456 bytes in size.

### TECHNICAL FIELD OF THE INVENTION

The present invention relates to a method of treating a subject suffering from a chronic inflammatory pain by administering to the subject a dose of anti-interleukin-36 receptor (anti-IL-36R) antibody such that the treatment results in a reduction or elimination of pain in the subject. More specifically, the present invention relates to treating a patient suffering from a chronic inflammatory pain by administering to the patient a dose of spesolimab effective to reduce the chronic inflammatory pain.

### BACKGROUND

The anti-IL-36 receptor (IL-36R) antibodies including spesolimab (B1655130) reduce or block IL-36 ligand-mediated signaling and are useful in treating diseases or conditions associate with such signaling.

Interleukin 36 (IL-36) is a group of cytokines in the IL-1 family with pro-inflammatory effect. There are four members of the IL-36 family, IL-36α (IL-1F6), IL-36β (IL-1F8), IL-36γ (IL-1F9) and IL-36Ra (IL-1F5), which all bind to IL-36R (previously referred to as IL-1 Rrp2) forming a heterodimer with IL-1 RAcP. IL-36R ligands are involved in a number of autoimmune and inflammatory diseases and conditions, such as inflammatory bowel disease (IBD), Crohn's disease (CD), ulcerative colitis (UD), atopic dermatitis (AtD), palmoplantar pustulosis (PPP), generalized pustular psoriasis (GPP) and neutrophilic dermatosis.

Emerging evidence indicates that spinal neuroinflammation contributes to the maintenance of chronic inflammatory pain. IL-36γ and IL-36R, but not IL-36α and IL-36β, were persistently upregulated in the spinal cord of mice with intraplantar injections of complete Freund's adjuvant (CFA). Intrathecal administration of both IL-36R antagonist (IL-36Ra) and IL-36γ siRNA significantly attenuated CFA-induced chronic inflammatory pain behaviors. These findings revealed that the neuronal/astrocytic interaction in the spinal cord by which neuronally produced IL-36γ activates astrocytes via IL-36R-mediated JNK pathway is crucial for the maintenance of chronic inflammatory pain. (Li Q. et al., Glia 67:3, 438-451, 2019)

Chronic inflammatory pain is a common symptom of a variety of autoimmune and inflammatory diseases and pathologic conditions, and includes nociceptive pain (related to an injury to body tissues caused by an inflammatory disease or condition), neuropathic pain (related to abnormalities in the nerves, spinal cord, or brain as a result of an inflammatory disease or condition), and psychogenic pain (entirely or mostly related to a psychological effects of an inflammatory disease or condition). Nociceptive pain includes somatic pain, which arises from bone, joint, muscle, skin, or connective tissue, and visceral pain, which arises from visceral organs, such as the gastrointestinal tract and the pancreas.

Mild chronic inflammatory pain is typically treated by nonsteroidal anti-inflammatory drugs (NSAIDs), such as acetaminophen, ibuprofen, aspirin, ketorolac, etodolac, and the like. Treatment of moderate to severe chronic inflammatory pain often includes opiate and NSAID combinations, such as aspirin and oxycodone (Percodan), acetaminophen and hydrocodone (Vicodin and Lortab). However, long term treatment of chronic inflammatory pain with NSAIDs or opiates are not optimal because of common side effects which include drowsiness, gastrointestinal problems, tolerance and drug dependency.

Moderate to severe, persisting pain is debilitating for patients with autoimmune and inflammatory diseases and conditions, and is often under-treated due to the side effects associated with long term use of NSAIDs and opiates. Since current therapies are unsatisfactory, it is important to develop further treatment modalities for the management of chronic inflammatory pain, that are more effective and are devoid of the undesired side-effects and risks associated with current treatment approaches.

### SUMMARY OF THE INVENTION

This invention is based, at least in part, on the surprising observation that patients with PPP treated with an IL-36R antibody, namely spesolimab, experienced a rapid reduction in pain, even where the visual symptoms associated with PPP in these patients were still present. This indicates that an IL-36R antibody, particularly spesolimab, has analgesic properties against chronic inflammatory pain.

In one aspect, the present invention relates to a method of treating a subject suffering from a chronic inflammatory pain, said method comprises administering to the subject a dose of spesolimab wherein the treatment results in a reduction or elimination of the chronic inflammatory pain in the subject.

In an embodiment relating to the above aspect, the chronic inflammatory pain is measured by a pain score or a quality of life score reflective of pain or its impact. In a related embodiment, the chronic inflammatory pain is measured by a visual analog scale (VAS) of 0-100 reflective of the subjective feeling of pain of the subject or by a numeric rating scale (NRS) of 0-10 reflective the subjective intensity or severity of pain of the subject.

In an embodiment relating to the above aspect or any of the above embodiments, the chronic inflammatory pain is measured once every few weeks (e.g., at week 1, 4, 8, 12, 16), weekly or daily. In a related embodiment, the chronic inflammatory pain is selected from the group consisting of nociceptive pain, neuropathic pain and psychogenic pain.

In an embodiment relating to the above aspect or any of the above embodiments, the chronic inflammatory pain is associated with an autoimmune and inflammatory disease or condition. In a related embodiment, the autoimmune and inflammatory condition comprises multiple sclerosis, asthma, type 1 diabetes mellitus, rheumatoid arthritis, scleroderma, Crohn's disease, psoriasis vulgaris (commonly referred to as psoriasis), pustular psoriasis, generalized pustular psoriasis (GPP), palmo-plantar pustulosis (PPP), inflammatory bowel disease, psoriatic arthritis, multiple sclerosis, rheumatoid arthritis, systemic lupus erythematosus (SLE), ulcerative colitis, ankylosing spondylitis, neutrophilic dermatoses, hidradenitis suppurativa (HS), Netherton syndrome (NS), allergic inflammation of the skin, lungs, and gastrointestinal tract, atopic dermatitis (also known as atopic eczema), asthma (allergic and non-allergic), epithelial-mediated inflammation, fibrosis (e.g., idiopathic pulmonary fibrosis, scleroderma, kidney fibrosis, and scarring), allergic rhinitis, food allergies (e.g., allergies to peanuts, eggs, dairy, shellfish, tree nuts, etc.), seasonal allergies, or other allergies. In a related embodiment, the chronic inflammatory pain is associated with PPP. In a related embodiment, the chronic inflammatory pain is associated with GPP.

In an embodiment relating to the above aspect or any of the above embodiments, the chronic inflammatory pain is selected from the group consisting of hand pain, foot pain, muscular pain, muscular tenderness, sharp pain, joint pain, neck pain, back pain, hip pain, pain from pustular lesions, pain from skin fissures or cracks, pain from desquamation, pain from erythema, burning pain, soreness, pain with stinging sensation, pain-associated discomfort, bodily pain, headache and pain associated with standing, walking, running, or climbing up or down stairs.

In another embodiment relating to the above aspect or any of the above embodiments, the dose of spesolimab is selected from the group consisting of 150 mg, 300 mg, 450 mg, 600 mg, 750 mg, 900 mg, 1050 mg, 1200 mg. In a related embodiment, the dose of spesolimab is administered intravenously or subcutaneously. In another related embodiment, the dose of spesolimab is administered at a qw (once every week), q2w (once every 2 weeks), q4w (once every 4 weeks), q6w (once every 6 weeks), q8w (once every 8 weeks), or q12w (once every 12 weeks) interval, or a combination thereof.

In an embodiment relating to the above aspect or any of the above embodiments, the reduction of the chronic inflammatory pain as measured by VAS or NRS is between 5%-60% or at least 10%, 20%, 30%, 40%, 50% or 60% at one, two, three, four, eight or twelve weeks after the treatment with spesolimab commences. In a related embodiment, the reduction of the chronic inflammatory pain as measured by VAS or NRS is by at least 1, or by at least 2, or by at least 3 grades, and results in a pain score (on a 0 to 10 scale) of no more than 5, or no more than 4, or no more than 3, or no more than 2 after one, two, three, four or eight or twelve weeks of treatment with the anti-IL-36R antibody (e.g., spesolimab).

In one aspect, the present invention relates to a method for treating a chronic inflammatory pain in a subject, comprising administering to the subject a therapeutically effective amount of an anti-IL-36R antibody or an antigen-binding fragment thereof (as disclosed herein), wherein the treatment results in a reduction or elimination of the chronic inflammatory pain in the subject.

In one aspect, the present invention relates to a method of treating pain associated with an autoimmune and inflammatory disease or condition in a subject, said method including administering or having administered to the subject a therapeutically effective amount of an anti-IL-36R antibody or an antigen binding fragment thereof (as disclosed herein), wherein the treatment results in a reduction or elimination of the chronic inflammatory pain in the subject.

In one aspect, the present invention relates to a method of treating a subject suffering from a chronic inflammatory pain, said method comprises (a) measuring the chronic inflammatory pain in the subject by a visual analog scale (VAS) of 0-100 reflective of the subjective feeling of pain of the subject or by a numeric rating scale (NRS) of 0-10 reflective the subjective intensity or severity of pain of the subject, (b) administering to the subject a dose of spesolimab if the VAS in the subject is greater than 30 or 40 or 50 or 60, or if the NRS in the subject is greater than 3 or 4 or 5 or 6, wherein the treatment results in a reduction or elimination of the chronic inflammatory pain in the subject, and wherein the reduction of the chronic inflammatory pain as measured by NRS is by at least 1, or by at least 2, or by at least 3 grades, and results in a pain score (on a 0 to 10 scale) of no more than 5, or no more than 4, or no more than 3, or no more than 2 after four or eight or twelve or sixteen or fifty-two weeks of treatment with spesolimab, or wherein the reduction of the chronic inflammatory pain as measured by VAS is by at least 5, or by at least 10, or by at least 15 points, and results in a pain VAS score (on a 0 to 100 scale) of no more than 90, or no more than 80, or no more than 70, or no more than 60, or no more than 50 after four or eight or twelve or sixteen or fifty-two weeks of treatment with spesolimab.

In an embodiment relating to any of the above aspects, a second therapeutic agent is administered to the subject before, after, or concurrent with the anti-IL-36R antibody or an antigen-binding fragment thereof. In a related embodiment, the second therapeutic agent comprises another IL-36R antagonist or an NSAID.

In an embodiment relating to any of the above aspects, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 or 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the anti-IL-36R antibody includes:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VII. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the anti-IL-36R antibody includes:
(i) a light chain variable region comprising the amino acid sequence of SEQ ID NO: 77; and a heavy chain variable region comprising the amino acid sequence of SEQ ID NO: 87; or
(ii) a light chain variable region comprising the amino acid sequence of SEQ ID NO: 77; and a heavy chain variable region comprising the amino acid sequence of SEQ ID NO: 88; or
(iii) a light chain variable region comprising the amino acid sequence of SEQ ID NO: 77; and a heavy chain variable region comprising the amino acid sequence of SEQ ID NO: 89; or
(iv) a light chain variable region comprising the amino acid sequence of SEQ ID NO: 80; and a heavy chain variable region comprising the amino acid sequence of SEQ ID NO: 87; or
(v) a light chain variable region comprising the amino acid sequence of SEQ ID NO: 80; and a heavy chain variable region comprising the amino acid sequence of SEQ ID NO: 88; or
(vi) a light chain variable region comprising the amino acid sequence of SEQ ID NO: 80; and a heavy chain variable region comprising the amino acid sequence of SEQ ID NO: 89; or
(vii) a light chain variable region comprising the amino acid sequence of SEQ ID NO: 85; and a heavy chain variable region comprising the amino acid sequence of SEQ ID NO: 100; or
(viii) a light chain variable region comprising the amino acid sequence of SEQ ID NO: 85; and a heavy chain variable region comprising the amino acid sequence of SEQ ID NO:101; or
(ix) a light chain variable region comprising the amino acid sequence of SEQ ID NO: 86; and a heavy chain variable region comprising the amino acid sequence of SEQ ID NO: 100; or
(x) a light chain variable region comprising the amino acid sequence of SEQ ID NO: 86; and a heavy chain variable region comprising the amino acid sequence of SEQ ID NO:101.

In an embodiment relating to any of the above aspects, the anti-IL-36R antibody includes:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

In an embodiment relating to any of the above aspects and/or embodiments, the autoimmune and inflammatory disease or condition comprises multiple sclerosis, asthma, type 1 diabetes mellitus, rheumatoid arthritis, scleroderma, Crohn's disease, psoriasis vulgaris (commonly referred to as psoriasis), pustular psoriasis, generalized pustular psoriasis (GPP), palmo-plantar pustulosis (PPP), inflammatory bowel disease, psoriatic arthritis, multiple sclerosis, rheumatoid arthritis, systemic lupus erythematosus (SLE), ulcerative colitis, ankylosing spondylitis, neutrophilic dermatosis, hidradenitis suppurativa (HS), Netherton syndrome (NS), allergic inflammation of the skin, lungs, and gastrointestinal tract, atopic dermatitis (also known as atopic eczema), asthma (allergic and non-allergic), epithelial-mediated inflammation, fibrosis (e.g., idiopathic pulmonary fibrosis, scleroderma, kidney fibrosis, and scarring), allergic rhinitis, food allergies (e.g., allergies to peanuts, eggs, dairy, shellfish, tree nuts, etc.), seasonal allergies, or other allergies.

In an embodiment relating to any of the above aspects and/or embodiments, the autoimmune and inflammatory disease or condition comprises hidradenitis suppurativa (HS); acute generalized exanthematous pustulosis; acute febrile neutrophilic dermatosis (Sweet syndrome); amicrobial pustulosis of the folds (APF); Behcet disease; Bowel bypass syndrome (bowel-associated dermatitis-arthritis syndrome); bowel-associated dermatosis-arthritis syndrome (BADAS); CARD14-mediated pustular psoriasis (CAMPS); cryopyrin associated periodic syndromes (CAPS); deficiency of interleukin-36 receptor antagonist (DIRTA); deficiency of interleukin-I receptor antagonist (DIRA); erythema elevatum diutinum; Histiocytoid neutrophilic dermatitis; infantile acropustulosis; neutrophilic dermatosis of the dorsal hands; neutrophilic eccrine hidradenitis; neutrophilic urticarial dermatosis; palisading neutrophilic granulomatous dermatitis; plaque psoriasis; pyoderma gangrenosum, acne, and hidradenitis suppurativa (PASH) syndrome; pyoderma gangrenosum (PG); pyoderma gangrenosum and acne (PAPA); pyogenic arthritis; skin lesions of Behcet's disease; Still's disease; subcorneal pustulosis (Sneddon-Wilkinson); synovitis, acne, pustulosis-hyperostosis; ostcitis (SAPHO) syndrome; rheumatoid neutrophilic dermatitis (RND), and ichthyosis (and its subtypes including netherton syndrome or NS).

In another embodiment relating to any of the above aspects and/or embodiments described herein, the anti-IL-36R antibody is administered in the range of about 0.001 to about 1000 mg to a subject suffering from a neutrophilic dermatosis.

In an embodiment relating to any of the above aspects and/or embodiments, the reduction of the chronic inflammatory pain as measured by VAS or NRS is between 5%-60% or at least 10%, 20%, 30%, 40%, 50% or 60% at one, two, three, four, eight or twelve weeks after the treatment with the anti-IL-36R antibody (e.g., spesolimab) commences. In a related embodiment, the reduction of the chronic inflammatory pain as measured by VAS or NRS is by at least 1, or by at least 2, or by at least 3 grades, and results in a pain score (on a 0 to 10 scale) of no more than 5, or no more than 4, or no more than 3, or no more than 2 after four or eight or twelve weeks of treatment with the anti-IL-36R antibody (e.g., spesolimab).

In an embodiment relating to any of the above aspects and/or embodiments, the reduction of the chronic inflammatory pain as measured by VAS is by at least 5, or by at least 10, or by at least 15 points, and results in a pain VAS score (on a 0 to 100 scale) of no more than 90, or no more than 80, or no more than 70, or no more than 60, or no more than 50 after four or eight or twelve or sixteen or fifty-two weeks of treatment with spesolimab, or wherein the reduction of the chronic inflammatory pain as measured by NRS is by at least 1, or by at least 2, or by at least 3 grades, and results in a pain score (on a 0 to 10 scale) of no more than 5, or no more than 4, or no more than 3, or no more than 2 after four or eight or twelve or sixteen or fifty-two weeks of treatment with spesolimab.

It will be understood that any of the herein disclosed methods, administration schemes and/or dosing regimens also equally apply to the use of any of the disclosed IL36-R antibodies in such methods, administration schemes and/or dosing regimens: i.e. an anti IL36R antibody, as disclosed herein, for use in the treatment, prevention, reducing and/or amelioration of any of the disclosed diseases and/or conditions. In other words, the invention also provides for the use of an anti IL36R antibody, as disclosed herein, for the manufacture of a medicament for the treatment, prevention, reducing and/or amelioration of any of the disclosed diseases and/or conditions.

Additional features and advantages of the present invention will-become apparent from a review of the ensuing detailed description-set forth below, and in part will be apparent from the description, or may be learned by practice of the subject technology. It is to be understood that both the foregoing general description and the following detailed description are exemplary and explanatory and are intended to provide further explanation of the present invention as claimed.

### BRIEF DESCRIPTION OF THE FIGURES

The accompanying drawings, which are included to provide further understanding of the present invention and are incorporated in and constitute a part of this specification, illustrate aspects of the subject technology and together with the description serve to explain the principles of the present invention.

FIG. 1 shows the IL-36 antagonist ligands (IL-36RA/IL1F5, IL-38/ILF10) inhibiting the signaling cascade.

FIG. 2 shows the design of the study described in Example 2. *PPP ASI50 is defined as achieving a 50% decrease in PPP ASI from baseline. IV, intravenous; PPP ASI50, palmoplantar pustular Psoriasis Area and Severity Index 50; VAS, visual analog scale.

FIG. 3 shows the study disposition. *Last treatment administered at visit 10 (Week 12). †From the end of treatment until Week 32 (visit 13, end of trial).

FIG. 4 shows the mean (95% CI) percent change from baseline in PPP ASI total score over time. Full analysis set, observed cases. A hierarchical approach performed in order to control for multiplicity arising as a result of multiple treatment comparisons. CI, confidence interval; PPP ASI, Palmoplantar Pustular Area and Severity Index.

FIG. 5 shows the mean (95% CI) absolute change in pain VAS from baseline to Week 16 by treatment. Full analysis set, observed cases. CI, confidence interval; VAS, visual analog scale.

FIG. 6 shows the absolute change from baseline in PPP pain VAS up to week 52 in patients with PPP randomized to one of five treatment arms: High (loading 3000 mg at weeks 0 to 4, maintenance 600 mg q4w), Medium-high (loading 3000 mg, maintenance 300 mg q4w), Medium-low (loading 1500 mg, maintenance 600 mg q4w), Low (loading 1500 mg, maintenance 300 mg q4w initially then q8w), and Placebo & spesolimab (loading placebo, maintenance 600 mg (spesolimab) q4w starting at week 16) CI, confidence interval. VAS, visual analog scale.

### DETAILED DESCRIPTION OF THE INVENTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

In the following detailed description, numerous specific details are set forth to provide a full understanding of the present invention. It will be apparent, however, to one of ordinarily skilled in the art that the subject technology may be practiced without some of these specific details. In other instances, well-known structures and techniques have not been shown in detail so as not to obscure the present invention.

Unless defined otherwise, all technical and scientific terms used herein have the same meaning as commonly understood by one of ordinary skill in the art to which this invention belongs.

Without wishing to be bound by this theory, the inventors believe that an anti-IL-36R antibody, preferably spesolimab, is capable of rapidly reducing or eliminating pain associated with an autoimmune and inflammatory disease or condition even where other symptoms associated with such disease or condition improves gradually, remains unchanged or even worsen. This suggests that an IL-36R antibody, preferably spesolimab, is capable of treating chronic inflammatory pain in a patient independent of its effects on other symptoms associated an autoimmune and inflammatory disease or condition in the patient.

In one aspect, the present invention relates to a method for treating or reducing chronic inflammatory pain in a subject, comprising administering to the subject a therapeutically effective amount of an anti-IL-36R antibody or an antigen-binding fragment thereof. In an embodiment relating to this aspect, the anti-IL-36R antibody is spesolimab.

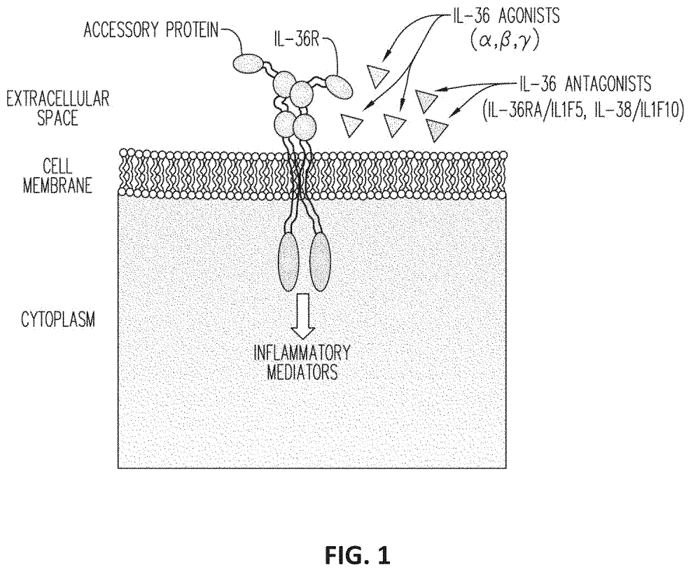

Without wishing to be bound by this theory it is believed that anti-IL-36R antibodies or antigen-binding fragments thereof bind to human IL-36R and thus interfere with the binding of IL-36 agonists, and in doing so block at least partially the signaling cascade from the IL-36R to pain mediators involved in autoimmune and inflammatory diseases or conditions. This is illustrated by FIG. 1. IL-36R is also known as IL-1 RL2 and IL-1 Rrp2. It has been reported that agonistic IL-36 ligands (α, β, or γ) initiate the signaling cascade by engaging the IL-36 receptor which then forms a heterodimer with the IL-1 receptor accessory protein (IL-1 RAcP).

### Definitions

A phrase such as “an aspect” does not imply that such aspect is essential to the present invention or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure.

The term “about” shall generally mean an acceptable degree of error or variation for the quantity measured given the nature or precision of the measurements. Typical, exemplary degrees of error or variation are within 5% or within 3% or within 1% of a given value or range of values. For example, the expression of “about 100” includes 105 and 95 or 103 and 97 or 101 and 99, and all values in between (e.g., 95.1, 95.2, etc. for range of 95-105; or 97.1, 97.2, etc. for the range of 97-103; 99.1, 99.2, etc. for the range of 99-101). Numerical quantities given herein are approximates unless stated otherwise, meaning that the term “about” can be inferred when not expressly stated.

A “pharmaceutical composition” refers in this context to a liquid or powder preparation which is in such form as to permit the biological activity of the active ingredient(s) to be unequivocally effective, and which contains no additional components which are significantly toxic to the subjects to which the composition would be administered. Such compositions are sterile.

The term “dose” as used herein refers to pharmaceutical composition containing an IL-36R antibody, preferably spesolimab, to be taken (e.g., orally or via intravenous or subcutaneous injection) at one time.

The term “subject” for purposes of treatment refers to any animal classified as a mammal, including humans, domesticated and farm animals, and zoo, sports, or pet animals, such as dogs, horses, cats, cows, and the like. Preferably, the mammal is human.

As used herein, the terms “treat”, “treating”, or the like, mean to alleviate symptoms, eliminate the causation of symptoms either on a temporary or permanent basis, or to prevent or slow the appearance of symptoms of the named disorder or condition. These terms are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an agent prior to or following the onset of a symptom of a disease or disorder thereby preventing or reducing the intensity of the symptom. As another example, the term includes the administration of an agent after clinical manifestation of the disease to combat the symptom(s) of the disease. Further, administration of an agent after onset and after clinical symptoms have developed where administration affects clinical parameters of the disease or disorder, such as the degree of pain, whether or not the treatment leads to amelioration of the underlying disease, comprises “treatment” or “therapy” as used herein.

The term “therapeutically effective amount” is used to refer to an amount of an active agent that reduces, relieves or ameliorates chronic inflammatory pain regardless of whether or not all other the symptoms associated with the underlying autoimmune and inflammatory disease or condition are alleviated. For example, the therapeutically effective amount refers to an amount or to a dose regimen that results in a target pain level of, e.g., 50 or less, 40 or less, 30 or less, as measured by a visual analog scale (VAS) of 0-100 or a target pain level of, e.g., 5 or less, 4 or less, 3 or less, as measured by a numeric rating scale (NRS) of 0-10.

As used herein, the term “chronic inflammatory pain” refers to pain caused by or associated with tissue injury resulting from an autoimmune and inflammatory disease or condition. Chronic inflammatory pain includes, without limitation, pain caused by or associated with arthritis, arthritic conditions, osteoarthritis, rheumatoid arthritis, gout, ankylosing spondylitis, multiple sclerosis, asthma, type 1 diabetes mellitus, scleroderma, Crohn's disease, psoriasis vulgaris (commonly referred to as psoriasis), pustular psoriasis, generalized pustular psoriasis (GPP), palmo-plantar pustulosis (PPP), inflammatory bowel disease, psoriatic arthritis, multiple sclerosis, rheumatoid arthritis, systemic lupus erythematosus (SLE), ulcerative colitis, allergic inflammation of the skin, lungs, and gastrointestinal tract, atopic dermatitis (also known as atopic eczema), asthma (allergic and non-allergic), epithelial-mediated inflammation, fibrosis (e.g., idiopathic pulmonary fibrosis, scleroderma, kidney fibrosis, and scarring), allergic rhinitis, food allergies (e.g., allergies to peanuts, eggs, dairy, shellfish, tree nuts, etc.), seasonal allergies, or other allergies, or neutrophilic dermatoses such as hidradenitis suppurativa (HS); acute generalized exanthematous pustulosis; acute febrile neutrophilic dermatosis (Sweet syndrome); amicrobial pustulosis of the folds (APF); Behcet disease; Bowel bypass syndrome (bowel-associated dermatitis-arthritis syndrome); bowel-associated dermatosis-arthritis syndrome (BADAS); CARD14-mediated pustular psoriasis (CAMPS); cryopyrin associated periodic syndromes (CAPS); deficiency of interleukin-36 receptor antagonist (DIRTA); deficiency of interleukin-I receptor antagonist (DIRA); erythema elevatum diutinum; Histiocytoid neutrophilic dermatitis; infantile acropustulosis; neutrophilic dermatosis of the dorsal hands; neutrophilic eccrine hidradenitis; neutrophilic urticarial dermatosis; palisading neutrophilic granulomatous dermatitis; plaque psoriasis; pyoderma gangrenosum, acne, and hidradenitis suppurativa (PASH) syndrome; pyoderma gangrenosum (PG); pyoderma gangrenosum and acne (PAPA); pyogenic arthritis; skin lesions of Behcet's disease; Still's disease; subcorneal pustulosis (Sneddon-Wilkinson); synovitis, acne, pustulosis-hyperostosis; ostcitis (SAPHO) syndrome; rheumatoid neutrophilic dermatitis (RND), and ichthyosis (and its subtypes including netherton syndrome or NS).

Although any methods and materials similar or equivalent to those described herein can be used in the practice of the present invention, the preferred methods and materials are now described. All publications mentioned herein are incorporated herein by reference to describe in their entirety.

IL36R is a novel member of the IL1R family that forms a heterodimeric complex with the IL1R accessory protein (IL1 RAcp) and IL1 Rrp2 associated with epithelial mediated inflammation and barrier dysfunction. The heterodimeric IL36R system with stimulating (IL36α, IL36β, IL36γ) and inhibitory ligands (IL36Ra and IL38) shares a number of structural and functional similarities to other members of the IL1/ILR family, such as IL1, IL18 and IL33. All IL1 family members (IL1α, IL1β, IL18, IL36α, IL36β, IL36γ, and IL38) signal through a unique, cognate receptor protein which, upon ligand binding, recruits the common IL1 RAcP subunit and activates NFγB and MAP kinase pathways in receptor-positive cell types (Dinarello, 2011; Towne et al., 2004; Towne et al., 2011).

The present invention is, at least partially, based on the finding that spesolimab, an anti-IL-36R antibody, exhibits analgesic effects in patients with PPP and is, therefore, useful in the management of chronic inflammatory pain. In particular, PPP patients treated with spesolimab reported a rapid reduction of pain even when their other PPP symptoms (e.g., number of pustules, skin fissures, cracks or erythema) were not reduced as quickly, remained the same or even worsen.

Therefore, in its broadest aspect, the invention relates to chronic inflammatory pain management using an anti-IL-36R antibody. In particular, the invention concerns the management of chronic inflammatory pain, either related to an autoimmune and inflammatory disease or condition or not associated with such a disease or condition, with an anti-IL-36R antibody, preferably spesolimab. The invention concerns the treatment of any type of chronic inflammatory pain, including, without limitation, nociceptive pain, somatic pain, visceral pain, neuropathic pain, centrally generated pain, and peripherally generated pain.

Given the connection between the IL36 pathway and chronic inflammatory pain, without wishing to be bound by this theory, it is believed that IL36R biology contributes to manifestation or maintenance of pain in autoimmune and inflammatory diseases or conditions and hence blocking IL36R activation will be beneficial in patients suffering from pain associated with such diseases or conditions.

Therefore, the present invention includes methods for treating a chronic inflammatory pain in a subject in need thereof, said method comprising administering a therapeutically effective amount of an anti-IL-36R antibody or an antigen binding fragment thereof to the subject such that the treatment results in improvement of pain the subject as measured by a pain assessment instrument such as VAS or NRS. As used herein, the expression “a subject in need thereof” means a human or a non-human animal that suffers from a chronic inflammatory pain.

In certain embodiments, the invention provides a method to reduce chronic inflammatory pain in patient by administering to the patient a dose of an anti-IL-36R antibody, wherein the treatment results in a reduction of pain (as measured by VAS or NRS) by at least 10%, at least 15%, at least 20%, at least 25%, at least 30%, at least 35%, at least 40%, at least 45%, at least 50%, at least 55%, at least 60%, at least 65%, at least 70%, or at least 75% at four, eight or twelve weeks after the treatment as compared to baseline (before the first administration of the anti-IL-36R antibody).

In one aspect, the present invention relates to a method of treating a subject suffering from a chronic inflammatory pain, said method comprises administering to the subject a dose of spesolimab wherein the treatment results in a reduction or elimination of the chronic inflammatory pain in the subject.

In an embodiment relating to the above aspect, the chronic inflammatory pain is measured by a pain score or a quality of life score reflective of pain or its impact. In a related embodiment, the chronic inflammatory pain is measured by a visual analog scale (VAS) of 0-100 reflective of the subjective feeling of pain of the subject or by a numeric rating scale (NRS) of 0-10 reflective the subjective intensity or severity of pain of the subject.

In an embodiment relating to the above aspect or any of the above embodiments, the chronic inflammatory pain is measured once every few weeks (e.g., at week 1, 4, 8, 12, 16), weekly or daily. In a related embodiment, the chronic inflammatory pain is selected from the group consisting of nociceptive pain, neuropathic pain and psychogenic pain.

In an embodiment relating to the above aspect and/or any of the above embodiments, the chronic inflammatory pain is associated with an autoimmune and inflammatory disease or condition. In a related embodiment, the autoimmune and inflammatory condition comprises multiple sclerosis, asthma, type 1 diabetes mellitus, rheumatoid arthritis, scleroderma, Crohn's disease, psoriasis vulgaris (commonly referred to as psoriasis), pustular psoriasis, generalized pustular psoriasis (GPP), palmo-plantar pustulosis (PPP), inflammatory bowel disease, psoriatic arthritis, multiple sclerosis, rheumatoid arthritis, systemic lupus erythematosus (SLE), ulcerative colitis, ankylosing spondylitis, neutrophilic dermatoses, hidradenitis suppurativa (HS), Netherton syndrome (NS), allergic inflammation of the skin, lungs, and gastrointestinal tract, atopic dermatitis (also known as atopic eczema), asthma (allergic and non-allergic), epithelial-mediated inflammation, fibrosis (e.g., idiopathic pulmonary fibrosis, scleroderma, kidney fibrosis, and scarring), allergic rhinitis, food allergies (e.g., allergies to peanuts, eggs, dairy, shellfish, tree nuts, etc.), seasonal allergies, or other allergies. In a related embodiment, the chronic inflammatory pain is associated with PPP. In a related embodiment, the chronic inflammatory pain is associated with GPP.

In an embodiment relating to the above aspect and/or any of the above embodiments, the chronic inflammatory pain is selected from the group consisting of hand pain, foot pain, muscular pain, muscular tenderness, sharp pain, joint pain, neck pain, back pain, hip pain, pain from pustular lesions, pain from skin fissures or cracks, pain from desquamation, pain from erythema, burning pain, soreness, pain with stinging sensation, pain-associated discomfort, bodily pain, headache and pain associated with standing, walking, running, or climbing up or down stairs.

In another embodiment relating to the above aspect and/or any of the above embodiments, the dose of spesolimab is selected from the group consisting of 150 mg, 300 mg, 450 mg, 600 mg, 750 mg, 900 mg, 1050 mg, 1200 mg. In a related embodiment, the dose of spesolimab is administered intravenously or subcutaneously. In another related embodiment, the dose of spesolimab is administered at a qw (once every week), q2w (once every 2 weeks), q4w (once every 4 weeks), q6w (once every 6 weeks), q8w (once every 8 weeks), or q12w (once every 12 weeks) interval, or a combination thereof.

In an embodiment relating to the above aspect and/or any of the above embodiments, the reduction of the chronic inflammatory pain as measured by VAS or NRS is between 5%-60% or at least 10%, 20%, 30%, 40%, 50% or 60% at one, two, three, four, eight or twelve weeks after the treatment with spesolimab commences. In a related embodiment, the reduction of the chronic inflammatory pain as measured by VAS or NRS is by at least 1, or by at least 2, or by at least 3 grades, and results in a pain score (on a 0 to 10 scale) of no more than 5, or no more than 4, or no more than 3, or no more than 2 after four or eight or twelve weeks of treatment with the anti-IL-36R antibody (e.g., spesolimab).

In one aspect, the present invention relates to a method for treating a chronic inflammatory pain in a subject, comprising administering to the subject a therapeutically effective amount of an anti-IL-36R antibody or an antigen-binding fragment thereof (as disclosed herein), wherein the treatment results in a reduction or elimination of the chronic inflammatory pain in the subject.

In one aspect, the present invention relates to a method of treating pain associated with an autoimmune and inflammatory disease or condition in a subject, said method including administering or having administered to the subject a therapeutically effective amount of an anti-IL-36R antibody or an antigen binding fragment thereof (as disclosed herein), wherein the treatment results in a reduction or elimination of the chronic inflammatory pain in the subject.

In an embodiment relating to any of the above aspects, a second therapeutic agent is administered to the subject before, after, or concurrent with the anti-IL-36R antibody or an antigen-binding fragment thereof. In a related embodiment, the second therapeutic agent comprises another IL-36R antagonist or an NSAID.

In an embodiment relating to any of the above aspects, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 or 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the anti-IL-36R antibody includes:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VII. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the anti-IL-36R antibody includes:
(i) a light chain variable region comprising the amino acid sequence of SEQ ID NO: 77; and a heavy chain variable region comprising the amino acid sequence of SEQ ID NO: 87; or
(ii) a light chain variable region comprising the amino acid sequence of SEQ ID NO: 77; and a heavy chain variable region comprising the amino acid sequence of SEQ ID NO: 88; or
(iii) a light chain variable region comprising the amino acid sequence of SEQ ID NO: 77; and a heavy chain variable region comprising the amino acid sequence of SEQ ID NO: 89; or
(iv) a light chain variable region comprising the amino acid sequence of SEQ ID NO: 80; and a heavy chain variable region comprising the amino acid sequence of SEQ ID NO: 87; or
(v) a light chain variable region comprising the amino acid sequence of SEQ ID NO: 80; and a heavy chain variable region comprising the amino acid sequence of SEQ ID NO: 88; or
(vi) a light chain variable region comprising the amino acid sequence of SEQ ID NO: 80; and a heavy chain variable region comprising the amino acid sequence of SEQ ID NO: 89; or
(vii) a light chain variable region comprising the amino acid sequence of SEQ ID NO: 85; and a heavy chain variable region comprising the amino acid sequence of SEQ ID NO: 100; or
(viii) a light chain variable region comprising the amino acid sequence of SEQ ID NO: 85; and a heavy chain variable region comprising the amino acid sequence of SEQ ID NO:101; or
(ix) a light chain variable region comprising the amino acid sequence of SEQ ID NO: 86; and a heavy chain variable region comprising the amino acid sequence of SEQ ID NO: 100; or
(x) a light chain variable region comprising the amino acid sequence of SEQ ID NO: 86; and a heavy chain variable region comprising the amino acid sequence of SEQ ID NO:101.

In an embodiment relating to any of the above aspects, the anti-IL-36R antibody includes:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

In an embodiment relating to any of the above aspects and/or embodiments, the autoimmune and inflammatory disease or condition comprises multiple sclerosis, asthma, type 1 diabetes mellitus, rheumatoid arthritis, scleroderma, Crohn's disease, psoriasis vulgaris (commonly referred to as psoriasis), pustular psoriasis, generalized pustular psoriasis (GPP), palmo-plantar pustulosis (PPP), inflammatory bowel disease, psoriatic arthritis, multiple sclerosis, rheumatoid arthritis, systemic lupus erythematosus (SLE), ulcerative colitis, ankylosing spondylitis, neutrophilic dermatosis, hidradenitis suppurativa (HS), Netherton syndrome (NS), allergic inflammation of the skin, lungs, and gastrointestinal tract, atopic dermatitis (also known as atopic eczema), asthma (allergic and non-allergic), epithelial-mediated inflammation, fibrosis (e.g., idiopathic pulmonary fibrosis, scleroderma, kidney fibrosis, and scarring), allergic rhinitis, food allergies (e.g., allergies to peanuts, eggs, dairy, shellfish, tree nuts, etc.), seasonal allergies, or other allergies.

In an embodiment relating to any of the above aspects and/or embodiments, the autoimmune and inflammatory disease or condition comprises hidradenitis suppurativa (HS); acute generalized exanthematous pustulosis; acute febrile neutrophilic dermatosis (Sweet syndrome); amicrobial pustulosis of the folds (APF); Behcet disease; Bowel bypass syndrome (bowel-associated dermatitis-arthritis syndrome); bowel-associated dermatosis-arthritis syndrome (BADAS); CARD14-mediated pustular psoriasis (CAMPS); cryopyrin associated periodic syndromes (CAPS); deficiency of interleukin-36 receptor antagonist (DIRTA); deficiency of interleukin-I receptor antagonist (DIRA); erythema elevatum diutinum; Histiocytoid neutrophilic dermatitis; infantile acropustulosis; neutrophilic dermatosis of the dorsal hands; neutrophilic eccrine hidradenitis; neutrophilic urticarial dermatosis; palisading neutrophilic granulomatous dermatitis; plaque psoriasis; pyoderma gangrenosum, acne, and hidradenitis suppurativa (PASH) syndrome; pyoderma gangrenosum (PG); pyoderma gangrenosum and acne (PAPA); pyogenic arthritis; skin lesions of Behcet's disease; Still's disease; subcorneal pustulosis (Sneddon-Wilkinson); synovitis, acne, pustulosis-hyperostosis; ostcitis (SAPHO) syndrome; rheumatoid neutrophilic dermatitis (RND), and ichthyosis (and its subtypes including netherton syndrome or NS).

In another embodiment relating to any of the above aspects and/or embodiments described herein, the anti-IL-36R antibody is administered in the range of about 0.001 to about 1000 mg to a subject suffering from a neutrophilic dermatosis.

In an embodiment relating to any of the above aspects and/or embodiments, the reduction of the chronic inflammatory pain as measured by VAS or NRS is between 5%-60% or at least 10%, 20%, 30%, 40%, 50% or 60% at one, two, three, four, eight or twelve weeks after the treatment with the anti-IL-36R antibody (e.g., spesolimab) commences. In a related embodiment, the reduction of the chronic inflammatory pain as measured by VAS or NRS is by at least 1, or by at least 2, or by at least 3 grades, and results in a pain score (on a 0 to 10 scale) of no more than 5, or no more than 4, or no more than 3, or no more than 2 after four or eight or twelve weeks of treatment with the anti-IL-36R antibody (e.g., spesolimab).

### Antibodies of the Present Invention

The anti-IL36R antibodies of the present invention are disclosed in U.S. Pat. No. 9,023,995 or WO2013/074569, the entire content of each of which is incorporated herein by reference.

The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-IL-36R antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

The antibodies used in the methods of the present invention may be human antibodies. The term “human antibody,” as used herein, is intended to include antibodies having variable and constant regions derived from human germline immunoglobulin sequences. The human antibodies of the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present invention may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

According to certain embodiments, the antibodies used in the methods of the present invention specifically bind IL-36R. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-36R, as used in the context of the present invention, includes antibodies that bind IL-36R or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human IL-36R may, however, have cross-reactivity to other antigens, such as IL-36R molecules from other (non-human) species.

In certain exemplary embodiments related to any aspects of the present invention, the anti-IL-36R antibody or antigen-binding fragment thereof that can be used in the context of the methods of the present invention includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 or 141 (H-CDR1); the amino acid sequence of SEQ ID NO: 62, 108, 109, 110, 111 or 142 (H-CDR2); the amino acid sequence of SEQ ID NO: 72 (H-CDR3).

According to certain embodiments, the anti-IL-36R antibody or antigen-binding fragment thereof comprises:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3) or
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VII. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

According to certain embodiments, the anti-IL-36R antibody or antigen-binding fragment thereof comprises:
(i) a light chain variable region comprising the amino acid sequence of SEQ ID NO: 77; and a heavy chain variable region comprising the amino acid sequence of SEQ ID NO: 87; or
(ii) a light chain variable region comprising the amino acid sequence of SEQ ID NO: 77; and a heavy chain variable region comprising the amino acid sequence of SEQ ID NO: 88; or
(iii) a light chain variable region comprising the amino acid sequence of SEQ ID NO: 77; and a heavy chain variable region comprising the amino acid sequence of SEQ ID NO: 89; or
(iv) a light chain variable region comprising the amino acid sequence of SEQ ID NO: 80; and a heavy chain variable region comprising the amino acid sequence of SEQ ID NO: 87; or
(v) a light chain variable region comprising the amino acid sequence of SEQ ID NO: 80; and a heavy chain variable region comprising the amino acid sequence of SEQ ID NO: 88; or
(vi) a light chain variable region comprising the amino acid sequence of SEQ ID NO: 80; and a heavy chain variable region comprising the amino acid sequence of SEQ ID NO: 89; or
(vii) a light chain variable region comprising the amino acid sequence of SEQ ID NO: 85; and a heavy chain variable region comprising the amino acid sequence of SEQ ID NO: 100; or
(viii) a light chain variable region comprising the amino acid sequence of SEQ ID NO: 85; and a heavy chain variable region comprising the amino acid sequence of SEQ ID NO:101; or
(ix) a light chain variable region comprising the amino acid sequence of SEQ ID NO: 86; and a heavy chain variable region comprising the amino acid sequence of SEQ ID NO: 100; or
(x) a light chain variable region comprising the amino acid sequence of SEQ ID NO: 86; and a heavy chain variable region comprising the amino acid sequence of SEQ ID NO:101.

According to certain embodiments, the anti-IL-36R antibody or antigen-binding fragment thereof comprises:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

In one aspect, described and disclosed herein are anti-IL-36R antibodies, in particular humanized anti-IL-36R antibodies, and compositions and articles of manufacture comprising one or more anti-IL-36R antibody, in particular one or more humanized anti-IL-36R antibody of the present invention. Also described are binding agents that include an antigen-binding fragment of an anti-IL-36 antibody, in particular a humanized anti-IL-36R antibody.

The term “spesolimab” as used herein refers to a humanized monoclonal IgG1 anti-IL-36R antibody having the [INN] name spesolimab, also registered under the CAS Registry Number 2097104-58-8.

### Pain Measurement

Pain measurement is essential for assessing analgesia. In human clinical trials, the pain experienced by an individual patient is assessed by using one or more of the known measures of pain, including a Visual Analog Scale (VAS), descriptive scale, numeric scale, Health Assessment Questionnaire (HAQ) pain index, Numeric Rating Scale (NRS) and the like. Table 1 below shows example pain assessment tools used in clinical trials.

TABLE 1
Pain Assessment Instruments

| | | Recall | |
|---|---|---|---|
| PRO | Pain Item | Period | Response Options |
| Pain VAS | Pain VAS for specific | Past | 0 (no pain) to 100 |
| | areas in the body: | week | (severe pain) |
| | “How much pain have | | |
| | you had because of your | | |
| | condition on, e.g., the | | |
| | palms and/or soles in | | |
| | the past week?” | | |
| | Pain VAS for muscular | | |
| | or joint pain: “How | | |
| | much muscular or joint | | |
| | pain have you had in | | |
| | the past week?” | | |
| Pain VAS | “How much pain have | Past | 0 (no pain) to 100 |
| | you had because of | week | (severe pain) |
| | your autoimmune and | | |
| | inflammatory | | |
| | disease/condition in the | | |
| | past week? | | |
| Pain NRS | Please rate the severity | Past 24 | 0 (no pain) to 10 |
| | of your condition's | hours | (worst possible pain) |
| | (e.g., palmoplantar | | |
| | pustulosis (PPP)) pain | | |
| | in the past 24 hours at | | |
| | its worst. | | |
| DLQI | Item 1: “Over the last | Past | “very much”, “a lot, |
| | week, how itchy, sore, | week | “a little”, and |
| | painful or stinging has | | “not at all” |
| | your skin been?” | | |
| PSS | “How severe was your | Past 24 | “none”, “mild”, |
| | pain from your | hours | “moderate”, “severe”, |
| | psoriasis during the | | and “very severe” |
| | past 24 hours?” | | |
| ppQLI | Pain dimension: | Past | “not painful”, “mildly |
| | “During the past month, | month | painful”, “moderately |
| | please indicate how | | painful”, “extremely |
| | painful, on average, | | painful”, and |
| | your foot/feet were | | “Could not do” |
| | during the following | | |
| | activities” | | |
| | Walking? | | |
| | Walking fast or running? | | |
| | Going up or down stairs? | | |
| | Standing barefoot? | | |
| | Standing wearing shoes? | | |
| PBI-S | Importance of | Current | “not at all”, |
| | treatment goal: | treatment | “somewhat”, |
| | “As a result of therapy, | | “moderately”, |
| | how important is it for | | “quite”, “very”, and |
| | you be free from pain?” | | “does not apply to me” |
| | Importance of | | |
| | treatment benefit: “The | | |
| | current treatment has | | |
| | helped me to be free | | |
| | from pain” | | |
| EQ-SD-SL | Pain/Discomfort: | Today | “I have no pain or |
| | Under each heading, | | discomfort” |
| | please tick the ONE box | | “I have slight pain or |
| | that best describes | | discomfort” |
| | your health TODAY. | | “I have moderate pain |
| | | | or discomfort” |
| | | | “I have severe pain or |
| | | | discomfort” |
| | | | “I have extreme pain or |
| | | | discomfort” |
| SF-36 | “How much bodily pain | Past | “none”, “very mild”, |
| | have you had during | week | “mild”, “moderate”, |
| | the past week?” | | “severe”, and “very |
| | | | severe” |
| | During the past week, | Past | “not at all”, |
| | how much did pain | week | “a little bit”, |
| | interfere with your | | “moderately”, “quite a |
| | normal work (including | | bit”, and “extremely” |
| | both work outside the | | |
| | home and housework?” | | |
| BASDAI | “How would you | Past 7 | 0 (none) to 10 |
| | describe your overall | days | (very severe) |
| | level of neck, back or | | |
| | hip pain resulting from | | |
| | AS?” | | |
| | “How would you | | |
| | describe your overall | | |
| | level of pain/swelling in | | |
| | joints other than the | | |
| | neck, back or hips?” | | |

The example pain assessment instruments that are used in clinical trials include Patient's Assessment of Arthritis Pain (PAAP/PtAAP VAS) (for indications, e.g., psoriatic arthritis or rheumatoid arthritis); Pain VAS (for indications, e.g., PGG, psoriatic arthritis, rheumatoid arthritis); Skin Pain VAS (for indications, e.g., plaque psoriasis); Joint Pain VAS (for indications, e.g., plaque psoriasis); Participant Assessment of Pain VAS (for indications, e.g., psoriatic arthritis, rheumatoid arthritis); Injection Site Pain VAS (for indications, e.g., arthritis, rheumatoid arthritis, psoriatic arthritis); Spinal Pain VAS (for indications, e.g., axial psoriatic arthritis); Reduction of Pain (VAS) (for indications, e.g., rheumatoid arthritis); Numerical Rating Scale (for indications, e.g., Pain Psoriasis, Psoriatic Arthritis); Brief Pain Inventory (NRS scales) (for indications, e.g., Osteoarthritis, Arthritis, Rheumatoid, Arthritis, Psoriatic); Subject Assessment of Pain (NRS) (for indications, e.g., Osteoarthritis, Arthritis, Rheumatoid, Arthritis, Psoriatic); Heel Pain Numerical Rating Scale (for indications, e.g., Chronic Scalp Psoriasis); Worst Pain Numeric Rating Scale (for indications, e.g., Psoriatic Arthritis, Axial Spondyloarthritis, Enthesitis); Numerical Rating Scale: Pain (for indications, e.g., Rheumatoid Arthritis)

In clinical trials across psoriasis and related indications, a VAS is commonly used as an endpoint to measure pain. VAS recall periods are, for example, Current/present, Past 24 hours, Past 7 days. Examples of Pain VAS anchor wording include 0 (no pain) to 100 (worst possible pain) or 0 (no pain) to 100 (most severe pain).

Most frequently, pain is assessed using a VAS recorded on various scales, such as a scale of 0 to 100, or 0 to 10, where the lowest score represents no pain, and the highest score represents the worst possible pain. Since it has been reported that patients have difficulty discriminating 100 levels of pain, the most frequently used VAS system operates on a scale of 0 to 10. The descriptive scale typically includes the following descriptions: no pain, mild pain, moderate pain, severe pain, very severe pain, and worst possible pain.

Additional guidelines/tools for assessing pain are provided by the U.S. Food & Drug administration in a document called Clinical Outcome Assessment (COA) Compendium. A copy of the COA Compendium can be found at www.fda.gov/drugs/development-resources/clinical-outcome-assessment—compendium (last viewed on September 2020), the entire content of which is incorporated herein by reference.

### Pharmaceutical Composition

An antibody of the invention can be incorporated into pharmaceutical compositions suitable for administration to a subject. The compounds of the invention may be administered alone or in combination with a pharmaceutically acceptable carrier, diluent, and/or excipients, in single or multiple doses. The pharmaceutical compositions for administration are designed to be appropriate for the selected mode of administration, and pharmaceutically acceptable diluents, carrier, and/or excipients such as dispersing agents, buffers, surfactants, preservatives, solubilizing agents, isotonicity agents, stabilizing agents and the like are used as appropriate.

A pharmaceutical composition comprising an anti-IL-36R monoclonal antibody of the present invention can be administered to a subject suffering from chronic inflammatory pain as described herein using standard administration techniques including oral, intravenous, intraperitoneal, subcutaneous, pulmonary, transdermal, intramuscular, intranasal, buccal, sublingual, or suppository administration.

The route of administration of an antibody of the present invention may be oral, parenteral, by inhalation, or topical. Preferably, the antibodies of the invention can be incorporated into a pharmaceutical composition suitable for parenteral administration. The term parenteral as used herein includes intravenous, intramuscular, subcutaneous, rectal, vaginal, or intraperitoneal administration. Peripheral systemic delivery by intravenous or intraperitoneal or subcutaneous injection is preferred. Suitable vehicles for such injections are known in the art.

The pharmaceutical composition typically must be sterile and stable under the conditions of manufacture arid storage in the container provided, including e.g., a sealed vial or syringe. Therefore, pharmaceutical compositions may be sterile filtered after making the formulation, or otherwise made microbiologically acceptable. A typical composition for intravenous infusion could have a volume as much as 250-1000 ml of fluid, such as sterile Ringer's solution, physiological saline, dextrose solution and Hank's solution and a therapeutically effective dose, (e.g., 1 to 100 mg/mL or more) of antibody concentration. Dose may vary depending on the type and severity of the disease. As is well known in the medical arts, dosages for any one subject depends upon many factors, including the patient's size, body surface area, age, the particular compound to be administered, sex, time and route of administration, general health, and other drugs being administered concurrently. A typical dose can be, for example, in the range of 0.001 to 1000 mg; however, doses below or above this exemplary range are envisioned, especially considering the aforementioned factors.

In an embodiment, a dose of spesolimab or the anti-IL-36R antibody used in the methods of the present invention is selected from the group consisting of 150 mg, 300 mg, 450 mg, 600 mg, 750 mg, 900 mg, 1050 mg, 1200 mg. In a related embodiment, the dose is administered intravenously or subcutaneously. In another related embodiment, the dose is administered at qw (once every week), q2w (once every 2 weeks), q4w (once every 4 weeks), q6w (once every 6 weeks), q8w (once every 8 weeks), or q12w (once every 12 weeks), or a combination thereof.

In another embodiment, a dose of spesolimab or the anti-IL-36R antibody used in the methods of the present invention is a pharmaceutical composition comprising:
I. about 20 mg/ml of the anti-IL-36R antibody, about 40 mM histidine, about 120 mM sucrose, about 50 mM L-Arginine, about 5 mM NaCl and about 1.0 g/L Polysorbate 20, with a pH of about 6.0;
II. about 60 mg/mL of the anti-IL-36R antibody, about 45 mM acetate, about 150 mM sucrose, about 25 mM L-Arginine, about 0.4 g/L Polysorbate 20, with a pH of about 5.5;
III. about 20 mg/mL of the anti-IL-36R antibody, about 45 mM acetate, about 180 mM sucrose, about 25 mM Glycine, about 0.4 g/L Polysorbate 80, with a pH of about 5.5;
IV. about 150 mg/mL of the anti-IL-36R antibody, about 25 mM citrate, about 150 mM trehalose, about 25 mM methionine, about 0.2 g/L Polysorbate 20, with a pH of about 6.0;
V. about 60 mg/mL of the anti-IL-36R antibody, about 25 mM histidine, about 160 mM sucrose, about 20 mM mannitol, about 0.2 g/L Polysorbate 20, with a pH of about 6.0;
VI. about 20 mg/mL of the anti-IL-36R antibody, about 25 mM citrate, about 200 mM sucrose, about 0.4 g/L Polysorbate 80, with a pH of about 6.5;
VII. about 150 mg/mL of the anti-IL-36R antibody, about 45 mM acetate, about 150 mM sucrose, about 25 mM L-Arginine, about 0.4 g/L Polysorbate 20, with a pH of about 5.5;
VIII. about 15 mg/mL of the anti-IL-36R antibody, about 35 mM histidine, about 180 mM trehalose, about 25 mM L-Arginine, about 3 mM NaCl, about 0.4 g/L Polysorbate 80, with a pH of about 6.0;
IX. about 80 mg/mL of the anti-IL-36R antibody, about 25 mM acetate, about 100 mM mannitol, about 50 mM NaCl, about 0.2 g/L Polysorbate 20, with a pH of about 5.5; or
X. about 100 mg/mL of the anti-IL-36R antibody, about 20 mM succinate, about 220 mM sucrose, about 0.1 g/L Polysorbate 80, with a pH of about 6.0.

### Chronic Inflammatory Pain Treatment

An anti-IL-36R antibody can be administered to human patients suffering from a chronic inflammatory pain in accord with known methods. Thus, for example, anti-IL-36R antibody (e.g. spesolimab) antibodies can be administered intravenously, e.g., as a bolus or by continuous infusion over a period of time, or by intramuscular, intraperitoneal, intracerobrospinal, subcutaneous, intra-articular, intrasynovial, intrathecal, oral, topical, or inhalation routes. Intravenous or subcutaneous administration of the antibodies is preferred.

Optimal dosage can be determined based upon dosing experiments in human clinical trials. Typically, an effective dose will reduce the patient's pain score (e.g., NRS or VAS) by at least 1, preferably by at least 2, more preferably by at least 3 grades, and preferably results in a pain score (on a 0 to 10 scale) of no more than 5, more preferably no more than 4, even more preferably no more than 3, most preferably no more than 2. Alternatively or in addition, an effective dose will reduce the patient's pain score (e.g., VAS) by at least 10, preferably by at least 20, more preferably by at least 30 grades, and preferably results in a pain score (on a 0 to 100 scale) of no more than 50, more preferably no more than 40, even more preferably no more than 30, most preferably no more than 20. The effective dose will also depend on the nature and severity of the initial pain.

The analgesic activity of a candidate anti-IL-36R antibody (e.g., spesolimab) is generally tested in a double-blind, randomized, placebo-controlled clinical trial.

Other therapeutic regimens may be combined with the administration of the anti-IL-36R antibody. The combined administration includes co-administration, using separate formulations or a single pharmaceutical formulation, and consecutive administration in either order, wherein preferably there is a time period while both (or all) active agents simultaneously exert their biological activities.

As discussed above, for the treatment of pain, the appropriate dosage of antibody will depend on the type of and severity of pain to be treated, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 mg/kg to 15 mg/kg (e.g. preferably about 0.1 or 0.5 to about 20 or about 30 mg/kg) of antibody is an initial candidate dosage for administration to the patient, whether, for example, by one or more separate administrations, or by continuous infusion. A typical daily dosage might range from about 1 mg/kg to 100 mg/kg or more, depending on the factors mentioned above. For repeated administrations over several days or longer, depending on the condition, the treatment is sustained until a desired reduction or suppression of pain occurs. The preferred dosage of the antibody will be in the range from about 0.5 mg/kg to about 30 mg/kg. Thus, one or more doses of about 0.5 mg/kg, 2.0 mg/kg, 4.0 mg/kg, 6 mg/kg, 8 mg/kg, 10 mg/kg or 15 mg/kg (or any combination thereof) may be administered to the patient. Such doses may be administered intermittently, e.g. every week, every three weeks, monthly or less frequently, for instance every 3 or 4 months (e.g. such that the patient receives from about two to about twenty, e.g. about six doses of the anti-IL-36R antibody). An initial higher loading dose, followed by one or more lower doses may be administered. An exemplary dosing regimen comprises administering an initial loading dose of about 900 mg an anti-IL-36R antibody administered weekly for four weeks, followed by a maintenance dose of about 600 mg of the anti-IL-36R antibody once every four weeks. However, other dosage regimens may be useful. The progress of this therapy is easily monitored by conventional techniques and assays.

Typically, an effective dose regimen of anti-IL-36R antibody (e.g., spesolimab) will reduce the patient's pain score (e.g., NRS or VAS) by at least 1, preferably by at least 2, more preferably by at least 3 grades, and preferably results in a pain score (on a 0 to 10 scale) of no more than 5, more preferably no more than 4, even more preferably no more than 3, most preferably no more than 2 after four, eight or twelve weeks of treatment with an anti-IL-36R antibody (e.g., spesolimab).

Accordingly, in one aspect, the present invention relates to a method for treating a chronic inflammatory pain in a subject, comprising administering to the subject a therapeutically effective amount of an anti-IL-36R antibody or an antigen-binding fragment thereof (as disclosed herein), wherein the treatment results in a reduction or elimination of the chronic inflammatory pain in the subject.

In one aspect, the present invention relates to a method of treating pain associated with an autoimmune and inflammatory disease or condition in a subject, said method including administering or having administered to the subject a therapeutically effective amount of an anti-IL-36R antibody or an antigen binding fragment thereof (as disclosed herein), wherein the treatment results in a reduction or elimination of the chronic inflammatory pain in the subject.

In one aspect, the present invention relates to a method of treating a subject suffering from a chronic inflammatory pain, said method comprises administering to the subject a dose of spesolimab wherein the treatment results in a reduction or elimination of the chronic inflammatory pain in the subject.

In an embodiment relating to the above aspect, the chronic inflammatory pain is measured by a pain score or a quality of life score reflective of pain or its impact. In a related embodiment, the chronic inflammatory pain is measured by a visual analog scale (VAS) of 0-100 reflective of the subjective feeling of pain of the subject or by a numeric rating scale (NRS) of 0-10 reflective the subjective intensity or severity of pain of the subject.

In an embodiment relating to the above aspect or any of the above embodiments, the chronic inflammatory pain is measured once every few weeks (e.g., at week 1, 4, 8, 12, 16), weekly or daily. In a related embodiment, the chronic inflammatory pain is selected from the group consisting of nociceptive pain, neuropathic pain and psychogenic pain.

In an embodiment relating to any of the above aspects and/or embodiments, the chronic inflammatory pain is reduced and the reduction of the chronic inflammatory pain as measured by VAS or NRS is between 5%-60% or at least 10%, 20%, 30%, 40%, 50% or 60% at one, two, three, four, eight or twelve weeks after the treatment with the anti-IL-36R antibody (e.g., spesolimab) commences. In a related embodiment, the reduction of the chronic inflammatory pain as measured by VAS or NRS is by at least 1, or by at least 2, or by at least 3 grades, and results in a pain score (on a 0 to 10 scale) of no more than 5, or no more than 4, or no more than 3, or no more than 2 after four or eight or twelve weeks of treatment with the anti-IL-36R antibody (e.g., spesolimab).

In an embodiment relating to any of the above aspects and/or embodiments, the chronic inflammatory pain is reduced and the reduction of the chronic inflammatory pain as measured by VAS is by at least 5, or by at least 10, or by at least 15 points, and results in a pain VAS score (on a 0 to 100 scale) of no more than 90, or no more than 80, or no more than 70, or no more than 60, or no more than 50 after four or eight or twelve or sixteen or fifty-two weeks of treatment with spesolimab, or wherein the reduction of the chronic inflammatory pain as measured by NRS is by at least 1, or by at least 2, or by at least 3 grades, and results in a pain score (on a 0 to 10 scale) of no more than 5, or no more than 4, or no more than 3, or no more than 2 after four or eight or twelve or sixteen or fifty-two weeks of treatment with spesolimab.

In one aspect, the present invention relates to a method of treating a subject suffering from a chronic inflammatory pain, said method comprises (a) measuring the chronic inflammatory pain in the subject by a visual analog scale (VAS) of 0-100 reflective of the subjective feeling of pain of the subject or by a numeric rating scale (NRS) of 0-10 reflective the subjective intensity or severity of pain of the subject, (b) administering to the subject a dose of spesolimab if the VAS in the subject is greater than 30 or 40 or 50 or 60, or if the NRS in the subject is greater than 3 or 4 or 5 or 6, wherein the treatment results in a reduction or elimination of the chronic inflammatory pain in the subject, and wherein the reduction of the chronic inflammatory pain as measured by NRS is by at least 1, or by at least 2, or by at least 3 grades, and results in a pain score (on a 0 to 10 scale) of no more than 5, or no more than 4, or no more than 3, or no more than 2 after four or eight or twelve or sixteen or fifty-two weeks of treatment with spesolimab, or wherein the reduction of the chronic inflammatory pain as measured by VAS is by at least 5, or by at least 10, or by at least 15 points, and results in a pain VAS score (on a 0 to 100 scale) of no more than 90, or no more than 80, or no more than 70, or no more than 60, or no more than 50 after four or eight or twelve or sixteen or fifty-two weeks of treatment with spesolimab.

### Articles of Manufacture

In another aspect, an article of manufacture containing materials useful for the treatment of the disorders described above is included. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port. For example, the container may be an intravenous solution bag or a vial having a stopper pierceable by a hypodermic injection needle. The active agent in the composition is the humanized anti-IL-36R antibody. The label on or associated with the container indicates that the composition is used for treating the condition of choice. The article of manufacture may further comprise a second container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use.

The invention is further described in the following examples, which are not intended to limit the scope of the invention.

### EXAMPLES

The following examples are intended to further illustrate certain preferred embodiments of the disclosure and are not limiting in nature. Those skilled in the art will recognize or be able to ascertain numerous equivalents to the specific substances and procedures described herein.

### Example 1: Treating Patients with Chronic Inflammatory Pain

In this example, an anti-IL36R antibody (e.g., spesolimab) is used to treat a patient with chronic inflammatory pain.

Following the administration of an effective dose regimen of the anti-IL-36R antibody, a pain assessment reveals that that patient's pain score (as measured by, e.g., NRS or VAS) is reduced by at least 1, preferably by at least 2, more preferably by at least 3 grades, and preferably results in a pain score (on a 0 to 10 scale) of no more than 5, more preferably no more than 4, even more preferably no more than 3, most preferably no more than 2 after four, eight or twelve weeks of treatment with the anti-IL-36R antibody (e.g., spesolimab).

### Example 2: Spesolimab, an IL-36R Inhibitor, Reduces Symptoms of Pain in Patients with Palmoplantar Pustulosis—Results from a Small Pilot Study

Palmoplantar pustulosis (PPP) is a chronic neutrophilic skin disease characterized by sterile neutrophil-filled pustules on the palms of the hands and soles of the feet, and PPP lesions can significantly impact patient quality of life. There has been little research on pain in PPP, and it may be under-appreciated by the dermatology community as it was reported as a debilitating symptom during patient focus groups. Our results suggested that spesolimab positively impact pain in patients with PPP.

This Phase IIa, multicenter, double-blind, randomized, placebo-controlled pilot study (NCT03135548) compared treatment with 900 mg spesolimab (n=19), 300 mg spesolimab (n=19), and placebo (n=21) intravenously every four weeks until Week 12, with follow-up to Week 32, in patients with PPP.

At baseline, patients reported considerable pain across the three groups. Mean (standard deviation [SD]) baseline pain visual analog scale (VAS) scores were: 900 mg: 67.95 (23.61); 300 mg: 58.42 (25.40); and placebo: 58.76 (28.23). Patients experienced a rapid and clinically meaningful (Olsen et al. J Clin Epidemiol 2018; 101:87-106) improvement in pain VAS from baseline to Week 16 (primary endpoint) with 900 mg spesolimab, with a mean (SD) absolute change of −32.67 (26.93) (n=15) compared with −0.81 (26.19) (n=16) with 300 mg spesolimab and −27.53 (26.09) (n=15) with placebo.

This pilot study suggested that although variability with spesolimab treatment is high, 900 mg spesolimab led to a rapid reduction in pain. A larger Phase IIb proof-of-concept and dose-finding study of spesolimab in patients with PPP is currently ongoing (NCT04015518).

Background: Palmoplantar pustulosis (PPP) is a chronic neutrophilic skin disease characterized by sterile neutrophil-filled pustules localized to the palms of the hands and soles of the feet. PPP lesions can have a significant impact on patients' quality of life. There has been little research on the pain experienced by patients with PPP, despite many patients reporting pain and a marked decrease (moderate-very severe impact) in quality of life due to pain associated with PPP. Often, this decrease in quality of life can be due to impaired function of the hands and feet, making everyday activities, such as walking, difficult to perform. Therapeutic intervention is often challenging in patients with PPP as patients often have little response to treatment. A lack of efficacy and the common occurrence of adverse events limits the length of the treatment course. Spesolimab is a humanized antagonistic monoclonal anti-interleukin-36 receptor (anti-IL-36R) antibody that blocks IL-36R signaling. In patients with generalized pustular psoriasis—a disease sharing similar pathologic features with PPP—spesolimab has previously been shown to result in rapid pustule clearance and marked improvements in other disease measures. Here, we present the results of a small pilot study investigating the effect of the treatment of PPP with spesolimab, an IL-36R inhibitor, on pain relief, as measured by the pain visual analog scale (VAS), a patient-reported outcome (PRO).

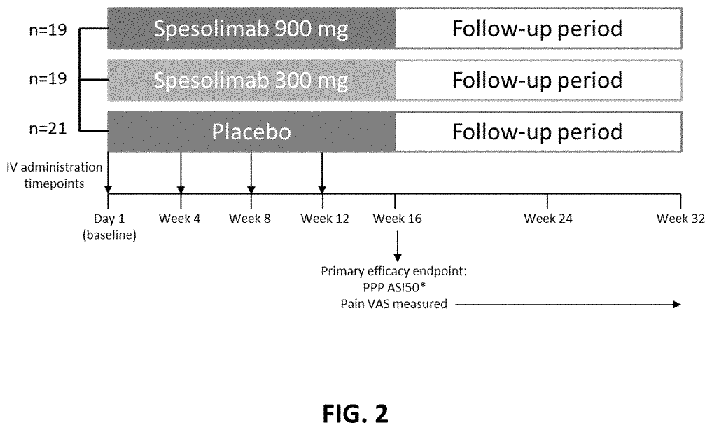

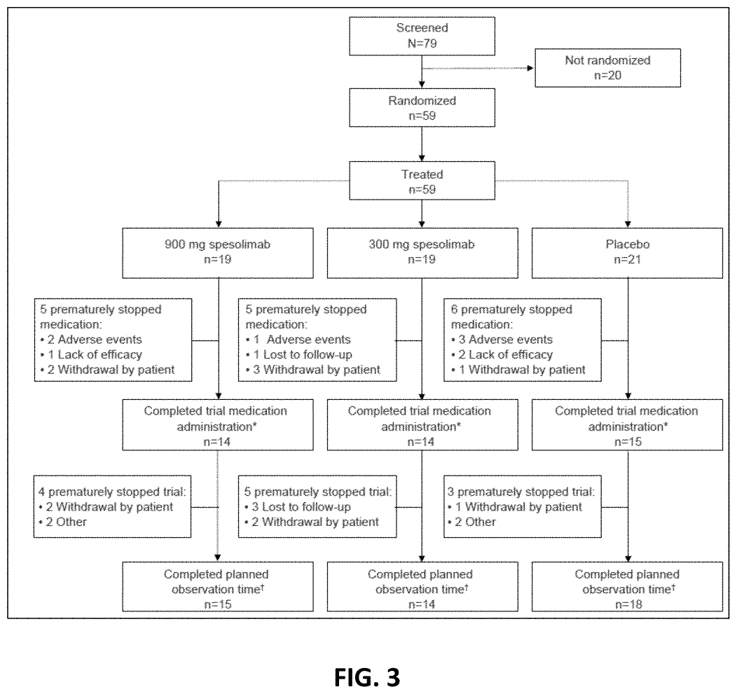

Methods: This study was a Phase IIa, multicenter, double-blind, randomized, placebo-controlled pilot study (NCT03135548). Patients were included if they had PPP, defined as the presence of primary, persistent (>3 months duration), sterile, macroscopically visible pustules on the palms of the hands and/or soles of the feet; 2 patients were permitted to have plaque psoriasis, providing it was not present on >10% of their body surface area. Patients were required to have active pustulation (yellow pustules), have a minimum Palmoplantar Pustular Psoriasis Area and Severity Index (PPP ASI) score of 12, and a Palmoplantar Pustulosis Physician Global Assessment (PPP PGA) of at least moderate severity (≥3) at baseline. A total of 59 patients were identified during screening and randomized 1:1:1, blinded, to one of two dose arms of spesolimab (900 mg [n=19] or 300 mg [n=19]) or placebo (n=21), administered intravenously every 4 weeks, corresponding to Day 1 and Weeks 4, 8, and 12 of treatment, with follow-up to Week 32 (FIG. 2, FIG. 3).

The primary endpoint in this study was the achievement of PPP ASI50 at Week 16. The main secondary endpoints were the achievement of PPP ASI75 at Week 16, percent change from baseline in the PPP ASI at Week 16 and treatment success defined as achieving a clinical response via the PPP Physician Global Assessment (PPP PGA) at Week 16. Further endpoints included: PPP ASI50, PPP ASI75, percent change from baseline in PPP ASI, and clinical response via PPP PGA at all other visits, time to achieving, or loss of, PPP ASI50, change in baseline in pain VAS at Week 16 and clinical improvement assessed by the Dermatology Life Quality Index at Week 16.

The pain VAS, a unidimensional measure of pain intensity, was measured at Week 16 and continued weekly for the duration of the study. Patients were asked, ‘How much pain have you had because of your PPP in the past week?’. The pain VAS was then self-completed by the patient, who placed an ‘X’ at the point on the horizontal line, on a scale of 0-100 (with 100 being the most pain imaginable), that most represented their pain intensity. Descriptive statistics were used to measure the absolute change and percent change from baseline over time in pain VAS in the full analysis set.

For the main primary endpoint and further endpoints presented here, an observed cases approach was used as a sensitivity analysis, including all collected data with no imputation performed on missing data. This approach excluded all values measured after intake of a rescue medication. The last observation carried forward was also used for percent change in pain VAS.

Results

Baseline demographics: Baseline demographics and disease characteristics were generally well-balanced between treatment arms (Table 2).

TABLE 2
Baseline demographics and disease characteristics

| | | | Spesolimab | | | | | | Total | |
|---|---|---|---|---|---|---|---|---|---|---|
| | Placebo | | 300 mg | | 900 mg | | Total | | overall | |
| | (n = 21) | | (n = 19) | | (n = 19) | | (n = 38) | | (N = 59) | |
| Sex, n (%) | | | | | | | | | | |
| Female | 17 | (81.0) | 16 | (84.2) | 16 | (84.2) | 32 | (84.2) | 49 | (83.1) |
| Male | 4 | (19.0) | 3 | (15.8) | 3 | (15.8) | 6 | (15.8) | 10 | (16.9) |
| Age, years | 46.3 ± 11.7 | | 54.6 ± 7.7 | | 49.4 ± 11.3 | | 52.0 ± 9.9 | | 50.0 ± 10.9 | |
| Race, n (%) | | | | | | | | | | |
| White | 19 | (90.5) | 18 | (94.7) | 19 | (100) | 37 | (97.4) | 56 | (94.9) |
| Asian | 1 | (4.8) | 0 | | 0 | | 0 | | 1 | (1.7) |
| Black | 1 | (4.8) | 0 | | 0 | | 0 | | 1 | (1.7) |
| Multiple | 0 | | 1 | (5.3) | 0 | | 1 | (2.6) | 1 | (1.7) |
| Weight, kg | 79.0 ± 15.8 | | 81.3 ± 12.7 | | 76.8 ± 19.2 | | 79.1 ± 16.3 | | 79.0 ± 15.9 | |
| BMI, kg/m² | 29.0 ± 5.5 | | 29.5 ± 5.2 | | 27.2 ± 5.9 | | 28.4 ± 5.6 | | 28.3 ± 5.5 | |
| PPP ASI at baseline | 18.5 ± 7.6 | | 20.3 ± 6.4 | | 16.9 ± 4.3 | | 18.6 ± 5.7 | | 18.6 ± 6.3 | |
| PPP ASI at screening | 18.5 ± 7.4 | | 16.5 ± 5.3 | | 16.2 ± 3.9 | | 16.4 ± 4.6 | | 17.1 ± 5.8 | |
| Time since first diagnosis | 6.7 ± 7.0 | | 9.5 ± 12.1 | | 11.2 ± 14.0 | | 10.4 ± 13.0 | | 9.1 ± 11.3 | |
| of PPP, years | | | | | | | | | | |
| PPP PGA, n (%) | | | | | | | | | | |
| 4 | 6 | (28.6) | 7 | (36.8) | 4 | (21.1) | 11 | (28.9) | 17 | (28.8) |
| 3 | 15 | (71.4) | 12 | (63.2) | 15 | (78.9) | 27 | (71.1) | 42 | (71.2) |

| | | | | | |
|---|---|---|---|---|---|
| Pain VAS | 58.8 ± 28.2 | 58.4 ± 25.4 | 68.0 ± 23.6 | 63.2 ± 24.7 | 61.6 ± 25.8 |
| C-reactive protein, mg/L | 4.8 ± 5.5 | 4.4 ± 2.7 | 4.3 ± 3.9 | 4.4 ± 3.3 | 4.5 ± 4.2 |

Safety analysis set.
All values are mean ± SD, unless otherwise stated.
BMI, body mass index; PPP, palmoplantar pustulosis; PPP ASI, Palmoplantar Pustular Psoriasis Area and Severity Index; PPP PGA; Palmoplantar Pustulosis Physician Global Assessment; SD, standard deviation; VAS, visual analog scale.

Of the 59 randomized patients, 43 (72.9%) completed trial medication administration, and all patients, regardless of whether they completed treatment or not, were followed up until Week 32. The rate of premature discontinuation was similar in all treatment groups. The most frequent reasons for treatment discontinuation included adverse events (10.2%) and patient withdrawal (10.2%). Six patients discontinued treatment due to worsening of disease (n=3) or lack of improvement (n=3).

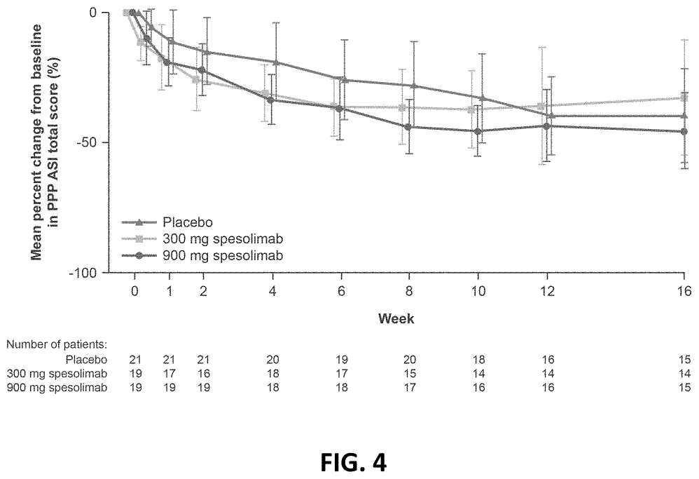

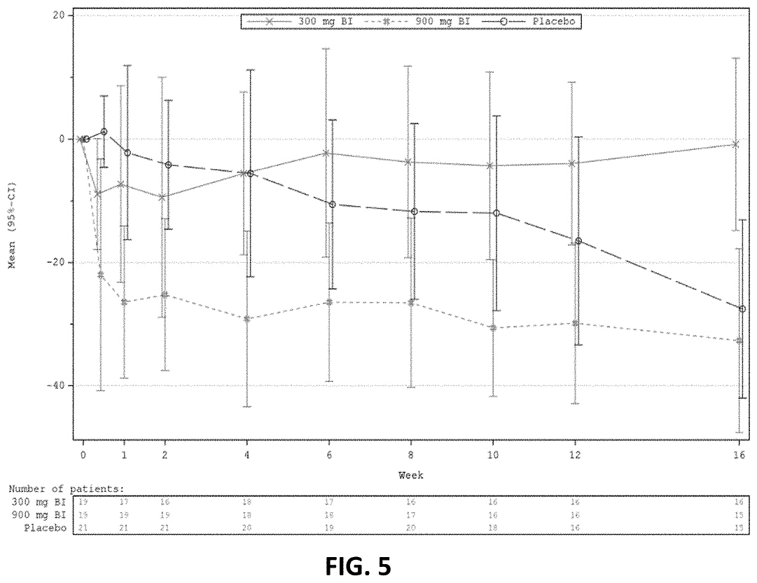

Change in pain VAS from baseline to Week 16: Overall, there was no significant change in the PPP ASI from baseline to Week 16 (FIG. 4), and the primary endpoint (PPP ASI50 at Week 16) was not met. The change in pain VAS from baseline to Week 16 was measured as a further endpoint. At baseline, patients reported considerable pain across all study arms. Mean (standard deviation [SD]) baseline pain VAS scores were: 900 mg spesolimab: 67.95 (23.61); 300 mg spesolimab: 58.42 (25.40); and placebo: 58.76 (28.23). Patients experienced a rapid and clinically meaningful improvement in pain VAS from baseline to Week 1 with 900 mg spesolimab (Day 8, absolute change from baseline [standard deviation, SD]: −26.42 [25.57]) (n=19) compared with 300 mg spesolimab (−7.29 [30.98]) (n=17) and placebo (−2.19 [30.99]) (n=21) (FIG. 5).

The reduction in pain was maintained from Week 1 through Week 16 with 900 mg spesolimab. The mean (SD) absolute change in pain VAS from baseline to Week 16 with 900 mg spesolimab was −32.67 (26.93) (n=15) compared with −2.80 (25.83) (n=15) with 300 mg spesolimab and −27.53 (26.09) (n=15) with placebo (FIG. 5).

Patients in the 900 mg spesolimab group also experienced the largest percent (SD) improvement in pain VAS from baseline to Week 16 (−48.06% [60.56]) compared with the 300 mg spesolimab group (pain VAS increased by 7.68% [43.93]) and the placebo group (−37.44% [72.29]). The largest reduction in pain was seen between baseline and Week 1 (Day 3) post treatment in the 900 mg spesolimab group (FIG. 5).

Discussion

There are currently a number of unmet clinical needs in patients with PPP, including the absence of a treatment with targeted efficacy that is not limited by a lack of response or the occurrence of adverse events. A particular unmet need in this patient population is the absence of a treatment that can effectively reduce the pain associated with PPP.

In this pilot study, the primary endpoint (PPP ASI50 at Week 16) was not met, with no significant differences between spesolimab and placebo observed. However, treatment with 900 mg spesolimab was associated with a rapid reduction in pain and warrants further investigation.

This pilot study was not without limitations, primarily that it was conducted in a relatively small sample of patients, and an effect in a small number of patients may have had a major proportional impact on the overall results. The primary endpoint at Week 16 may also have coincided with natural disease resolution in some patients.

### Example 3: A Multi-Center, Double-Blind, Randomized, Placebo-Controlled, Phase IIb Dose-Finding Study to Evaluate Efficacy and Safety of Spesolimab in Patients with Moderate-to-Severe Palmoplantar Pustulosis

Palmoplantar pustulosis (PPP) is a chronic inflammatory disease characterized by sterile pustules on the palms and soles, and has an impact on patient quality of life. Spesolimab is a first-in-class humanized anti-interleukin-36 receptor monoclonal IgG antibody, previously investigated in a Phase IIa PPP trial.

In this Phase IIb trial (NCT04015518), patients with moderate-to-severe PPP were assigned to one of five groups; they received a total subcutaneous loading dose of 1500/3000 mg spesolimab or placebo for the first 4 weeks followed by 300/600 mg spesolimab or placebo q4w. After Week 16, patients receiving placebo switched to spesolimab 600 mg q4w; maintenance with spesolimab continued q4w/q8w to Week 52. The primary endpoint was percent change in PPP Area and Severity Index (PPP ASI) from baseline at Week 16. Safety was assessed descriptively.

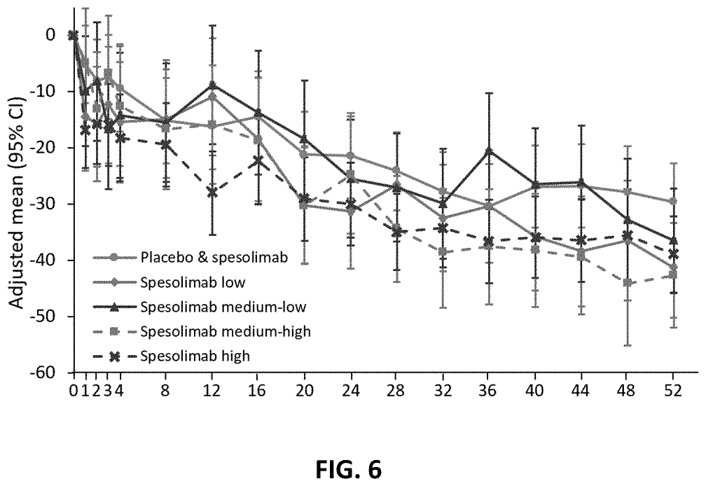

152 patients were randomized. At Week 16, no significant dose-response model was identified for the primary endpoint between spesolimab and placebo (spesolimab groups 1-4 combined: −43.3%; placebo: −33.6%); reduction in PPP ASI continued over 52 weeks in all groups. Efficacy assessed via PPP Physician's Global Assessment clear/almost clear was apparent at Weeks 16 (spesolimab vs. placebo: 21.1% vs. 4.7%) and 52 (spesolimab vs. placebo/spesolimab: 54.1% vs. 27.9%). Improvements in Dermatology Life Quality Index and pain visual analog scores were observed over 52 weeks. See FIG. 6 and Table 3 for Pain VAS results. Differences in efficacy favoring spesolimab over placebo/spesolimab were observed in non-Asian patients over 52 weeks. Spesolimab was generally well tolerated.

TABLE 4
Absolute change from baseline in PPP pain VAS up to Week
52 Mean (95% CI) of MMRM estimates - FAS (EC-MMRM)

| | Week 16 adj. | Week 52 adj. |
|---|---|---|
| | mean (SE) | mean (SE) |
| Placebo & spesolimab | −14.5 (4.1) | −29.6 (3.5) |
| Spesolimab low | −18.4 (5.6) | −41.3 (4.5) |
| Spesolimab medium-low | −13.7 (5.6) | −36.5 (4.7) |
| Spesolimab medium-high | −18.7 (5.6) | −42.7 (4.7) |
| Spesolimab high | −22.3 (4.0) | −39.0 (3.4) |

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

All patents and/or publications including journal articles cited in this disclosure are expressly incorporated herein by reference.

1. A method for treating a subject suffering from a chronic inflammatory pain, said method comprises administering to the subject a dose of spesolimab wherein the treatment results in a reduction or elimination of the chronic inflammatory pain in the subject.

2. The method of claim 1, wherein the chronic inflammatory pain is measured by a pain score or a quality of life score reflective of pain or its impact.

3. The method of claim 2, wherein the chronic inflammatory pain is measured by a visual analog scale (VAS) of 0-100 reflective of the subjective feeling of pain of the subject or by a numeric rating scale (NRS) of 0-10 reflective the subjective intensity or severity of pain of the subject.

4. The method of claim 1, wherein the chronic inflammatory pain is measured once every few weeks, weekly or daily.

5. The method of claim 1, wherein the chronic inflammatory pain is selected from the group consisting of nociceptive pain, neuropathic pain and psychogenic pain.

6. The method of claim 5, wherein the chronic inflammatory pain is associated with an autoimmune and inflammatory disease or condition.

7. The method of claim 6, wherein the autoimmune and inflammatory condition comprises multiple sclerosis, asthma, type 1 diabetes mellitus, rheumatoid arthritis, scleroderma, Crohn's disease, psoriasis vulgaris (commonly referred to as psoriasis), pustular psoriasis, generalized pustular psoriasis (GPP), palmo-plantar pustulosis (PPP), inflammatory bowel disease, psoriatic arthritis, multiple sclerosis, rheumatoid arthritis, systemic lupus erythematosus (SLE), ulcerative colitis, ankylosing spondylitis, neutrophilic dermatoses, hidradenitis suppurativa (HS), Netherton syndrome (NS), allergic inflammation of the skin, lungs, and gastrointestinal tract, atopic dermatitis (also known as atopic eczema), asthma (allergic and non-allergic), epithelial-mediated inflammation, fibrosis (e.g., idiopathic pulmonary fibrosis, scleroderma, kidney fibrosis, and scarring), allergic rhinitis, food allergies (e.g., allergies to peanuts, eggs, dairy, shellfish, tree nuts, etc.), seasonal allergies, or other allergies.

8. The method of claim 1, wherein the chronic inflammatory pain is associated with PPP.

9. The method of claim 1, wherein the chronic inflammatory pain is associated with GPP.

10. The method of claim 1, wherein the chronic inflammatory pain is selected from the group consisting of hand pain, foot pain, muscular pain, muscular tenderness, sharp pain, joint pain, neck pain, back pain, hip pain, pain from pustular lesions, pain from skin fissures or cracks, pain from desquamation, pain from erythema, burning pain, soreness, pain with stinging sensation, pain-associated discomfort, bodily pain, headache and pain associated with standing, walking, running, or climbing up or down stairs.

11. The method of claim 1, wherein the dose of spesolimab is selected from the group consisting of 150 mg, 300 mg, 450 mg, 600 mg, 750 mg, 900 mg, 1050 mg, 1200 mg.

12. The method of claim 1, wherein the dose of spesolimab is administered intravenously or subcutaneously.

13. The method of claim 1, wherein the dose of spesolimab is administered at a qw (once every week), q2w (once every 2 weeks), q4w (once every 4 weeks), q6w (once every 6 weeks), q8w (once every 8 weeks), or q12w (once every 12 weeks) interval, or a combination thereof.

14. The method of claim 3, wherein the reduction of the chronic inflammatory pain is at least 10% as measured by VAS or NRS four, eight or twelve weeks after the treatment with spesolimab commences.

15. The method of claim 3, wherein the reduction of the chronic inflammatory pain as measured by VAS is by at least 5, or by at least 10, or by at least 15 points, and results in a pain VAS score (on a 0 to 100 scale) of no more than 90, or no more than 80, or no more than 70, or no more than 60, or no more than 50 after four or eight or twelve or sixteen or fifty-two weeks of treatment with spesolimab, or wherein the reduction of the chronic inflammatory pain as measured by NRS is by at least 1, or by at least 2, or by at least 3 grades, and results in a pain score (on a 0 to 10 scale) of no more than 5, or no more than 4, or no more than 3, or no more than 2 after four or eight or twelve or sixteen or fifty-two weeks of treatment with spesolimab.

16. A method of treating a subject suffering from a chronic inflammatory pain, said method comprises (a) measuring the chronic inflammatory pain in the subject by a visual analog scale (VAS) of 0-100 reflective of the subjective feeling of pain of the subject or by a numeric rating scale (NRS) of 0-10 reflective the subjective intensity or severity of pain of the subject, (b) administering to the subject a dose of spesolimab if the VAS in the subject is greater than 30 or 40 or 50 or 60, or if the NRS in the subject is greater than 3 or 4 or 5 or 6, wherein the treatment results in a reduction or elimination of the chronic inflammatory pain in the subject, and wherein the reduction of the chronic inflammatory pain as measured by NRS is by at least 1, or by at least 2, or by at least 3 grades, and results in a pain score (on a 0 to 10 scale) of no more than 5, or no more than 4, or no more than 3, or no more than 2 after four or eight or twelve or sixteen or fifty-two weeks of treatment with spesolimab, or wherein the reduction of the chronic inflammatory pain as measured by VAS is by at least 5, or by at least 10, or by at least 15 points, and results in a pain VAS score (on a 0 to 100 scale) of no more than 90, or no more than 80, or no more than 70, or no more than 60, or no more than 50 after four or eight or twelve or sixteen or fifty-two weeks of treatment with spesolimab.


SEQUENCE LISTING (142 sequences)

SEQ ID NO: 1 - protein, 108 aa, Mus sp.
QIVLTQSPAIMSASLGERVTMTCTASSSVSSSYLHWYQKKPGSSPKLWVYSTSNLASGVPVRFSGSGSGTSYSLTISSMEAEDAATYYCHQHHRSPVTFGSGTKLEMK

SEQ ID NO: 2 - protein, 107 aa, Mus sp.
DIQMTQSPASQSASLGESVTFTCLASQTIGTWLAWYQQRPGKSPQLLIYAATSLADGVPSRFSGSGSGTQFSFNIRSLQAEDFASYYCQQVYTTPLTFGGGTKLEIK

SEQ ID NO: 3 - protein, 107 aa, Mus sp.
DIQMTQSPASQSASLGESVTFTCLASQTIGTWLGWYQQKPGKSPQLLIYRSTTLADGVPSRFSGSGSGTKFSFKISSLQAADFASYYCQQLYSAPYTFGGGTKLEIR

SEQ ID NO: 4 - protein, 112 aa, Mus sp.
DVLLTQTPLSLPVSLGDQASISCRSSQNIVHSNGNTYLQWYLQKPGQSPKLLIYKVSNRFSGVPDRFSGSGSGTDFTLKISRVEAEDLGVYYCFQGSHVPFTFGAGTKLELK

SEQ ID NO: 5 - protein, 107 aa, Artificial Sequence
  Description of Artificial Sequence Synthetic polypeptide
DIQMTQTTSSLSASLGDRVTISCRASQDIYKYLNWYQQKPDGTLKLLIYYTSGLHSGVPSRFSGSGSGTDFSLTISNLEPEDIATYFCQQDSKFPWTFGGDTKLEIK

SEQ ID NO: 6 - protein, 108 aa, Mus sp.
QIVLTQSPAIMSASLGERVTMTCTASSSVSSSYFHWYQQKPGSSPKLWIYRTSNLASGVPGRFSGSGSGTSYSLTISSMEAEDAATYYCHQFHRSPLTFGAGTKLELK

SEQ ID NO: 7 - protein, 107 aa, Mus sp.
DIVMTQSQKFLSTSVGVRVSVTCKASQDVGTNVLWYQQKIGQSPKPLIYSASYRHSGVPDRFTGSGSGTDFTLIISNVQSEDLAEYFCQQYSRYPLTFGPGTKLELK

SEQ ID NO: 8 - protein, 107 aa, Mus sp.
DIVMTQSQKFLSTSVGVRVSVTCKASQDVGTNVLWYQQKIGQSPKALIYSASYRHSGVPDRFTGSGSGTDFTLIITNVQSEDLAEYFCQQYSRYPLTFGPGTKLELK

SEQ ID NO: 9 - protein, 107 aa, Mus sp.
DIVMTQSQKFMSATVGGRVNITCKASQNVGRAVAWYQQKPGQSPKLLTHSASNRYTGVPDRFTGSGSGTDFTLTITNMQSEDLADYFCQQYSSYPLTFGAGTKLDLK

SEQ ID NO: 10 - protein, 107 aa, Mus sp.
DIQMTQSPASQSASLGESVTFSCLASQTIGTWLGWYQQKPGKSPQLLIYRATSLADGVPSRFSGSGSGTNFSFKISSLQAEDLASYYCQQLYSGPYTFGGGTKLEIR

SEQ ID NO: 11 - protein, 119 aa, Mus sp.
QVQLQQSGTELLKPGASVKLSCKASGNTVTSYWMHWVKQRPGQGLEWIGEILPSTGRTNYNENFKGKAMLTVDKSSSTAYMQLSSLASEDSAVYYCTIVYFGNPWFAYWGQGTLVTVSA

SEQ ID NO: 12 - protein, 125 aa, Mus sp.
EVQLQQSGPELVKPGASVKLSCKASGYTFTDNYMNWVRQSHGKSLEWIGRVNPSNGDTKYNQNFKGKATLTVDKSLSTAYMQLNGLTSEDSAVYYCGRTKNFYSSYSYDDAMDYWGQGTSVTVSS

SEQ ID NO: 13 - protein, 124 aa, Mus sp.
EVQLQQSGAEFVRPGASVKFSCTASGFNIKDDYIHWVRQRPEQGLEWVGRIDPANGNTKYAPKFQDKATITADTSSNTAYLQLSSLTSEDTAVYYCAKSFPNNYYSYDDAFAYWGQGTLVTVSA

SEQ ID NO: 14 - protein, 118 aa, Mus sp.
QVQLKESGPVLVAPSQSLSITCTVSGFSLTKFGVHWIRQTPGKGLEWLGVIWAGGPTNYNSALMSRLTISKDISQSQVFLRIDSLQTDDTAMYYCAKQIYYSTLVDYWGQGTSVTVSS

SEQ ID NO: 15 - protein, 120 aa, Mus sp.
QVQLKESGPGLVAPSQSLFITCTVSGFSLSSYEINWVRQVPGKGLEWLGVIWTGITTNYNSALISRLSISKDNSKSLVFLKMNSLQTDDTAIYYCARGTGTGFYYAMDYWGQGTSVTVSS

SEQ ID NO: 16 - protein, 119 aa, Mus sp.
QVQLQQPGADFVRPGASMRLSCKASGYSFTSSWIHWVKQRPGQGLEWIGEINPGNVRTNYNENFRNKATLTVDKSSTTAYMQLRSLTSADSAVYYCTVVFYGEPYFPYWGQGTLVTVSA

SEQ ID NO: 17 - protein, 119 aa, Mus sp.
QVQLKESGPGLVAPSQSLSITCTVSGFSLTNYAVHWVRQFPGKGLEWLGVIWSDGSTDFNAPFKSRLSINKDNSKSQVFFKMNSLQIDDTAIYYCARKGGYSGSWFAYWGQGTLVTVSA

SEQ ID NO: 18 - protein, 119 aa, Mus sp.
QVQLKESGPGLVAPSQSLSITCTVSGFSLTNYAVHWVRQFPGKGLEWLGVIWSDGSTDYNAPFKSRLSINKDNSKSQVFFKMNSLQTDDTAIYYCARKGGYSGSWFAYWGQGTLVTVSA

SEQ ID NO: 19 - protein, 117 aa, Mus sp.
QVQLKESGPVLVAPSQSLSITCTVSGFSLTNYGVHWVRQPPGKGLEWLGVIWPVGSTNYNSALMSRLSIHKDNSKSQVFLRMNSLQTDDTAIYYCAKMDWDDFFDYWGQGTTLTVSS

SEQ ID NO: 20 - protein, 124 aa, Mus sp.
EVQLQQSGAELVRPGASVRLSCTASGFNIKDDYIHWVRQRPKQGLEWLGRIDPANGNTKYDPRFQDKATITADTSSNTAYLHLSSLTSEDTAVYYCAKSFPDNYYSYDDAFAYWGQGTLVTVSA

SEQ ID NO: 21 - protein, 12 aa, Mus sp.
TASSSVSSSYLH

SEQ ID NO: 22 - protein, 11 aa, Mus sp.
LASQTIGTWLA

SEQ ID NO: 23 - protein, 11 aa, Mus sp.
LASQTIGTWLG

SEQ ID NO: 24 - protein, 16 aa, Mus sp.
RSSQNIVHSNGNTYLQ

SEQ ID NO: 25 - protein, 11 aa, Mus sp.
RASQDIYKYLN

SEQ ID NO: 26 - protein, 12 aa, Mus sp.
TASSSVSSSYFH

SEQ ID NO: 27 - protein, 11 aa, Mus sp.
KASQDVGTNVL

SEQ ID NO: 28 - protein, 11 aa, Mus sp.
KASQNVGRAVA

SEQ ID NO: 29 - protein, 11 aa, Mus sp.
LASQTIGTWLG

SEQ ID NO: 30 - protein, 7 aa, Mus sp.
STSNLAS

SEQ ID NO: 31 - protein, 7 aa, Mus sp.
AATSLAD

SEQ ID NO: 32 - protein, 7 aa, Mus sp.
RSTTLAD

SEQ ID NO: 33 - protein, 7 aa, Mus sp.
KVSNRFS

SEQ ID NO: 34 - protein, 7 aa, Mus sp.
YTSGLHS

SEQ ID NO: 35 - protein, 7 aa, Mus sp.
RTSNLAS

SEQ ID NO: 36 - protein, 7 aa, Mus sp.
SASYRHS

SEQ ID NO: 37 - protein, 7 aa, Mus sp.
SASNRYT

SEQ ID NO: 38 - protein, 7 aa, Mus sp.
RATSLAD

SEQ ID NO: 39 - protein, 9 aa, Mus sp.
HQHHRSPVT

SEQ ID NO: 40 - protein, 9 aa, Artificial Sequence
  Description of Artificial Sequence Synthetic peptide
QQVYTTPLT

SEQ ID NO: 41 - protein, 9 aa, Mus sp.
QQLYSAPYT

SEQ ID NO: 42 - protein, 9 aa, Mus sp.
FQGSHVPFT

SEQ ID NO: 43 - protein, 9 aa, Mus sp.
QQDSKFPWT

SEQ ID NO: 44 - protein, 9 aa, Mus sp.
HQFHRSPLT

SEQ ID NO: 45 - protein, 9 aa, Mus sp.
QQYSRYPLT

SEQ ID NO: 46 - protein, 9 aa, Mus sp.
QQYSSYPLT

SEQ ID NO: 47 - protein, 9 aa, Mus sp.
QQLYSGPYT

SEQ ID NO: 48 - protein, 10 aa, Mus sp.
GNTVTSYWMH

SEQ ID NO: 49 - protein, 10 aa, Mus sp.
GYTFTDNYMN

SEQ ID NO: 50 - protein, 10 aa, Mus sp.
GFNIKDDYIH

SEQ ID NO: 51 - protein, 10 aa, Mus sp.
GFSLTKFGVH

SEQ ID NO: 52 - protein, 10 aa, Mus sp.
GFSLSSYEIN

SEQ ID NO: 53 - protein, 10 aa, Mus sp.
GYSFTSSWIH

SEQ ID NO: 54 - protein, 10 aa, Mus sp.
GFSLTNYAVH

SEQ ID NO: 55 - protein, 10 aa, Mus sp.
GFSLTNYGVH

SEQ ID NO: 56 - protein, 10 aa, Mus sp.
GFNIKDDYIH

SEQ ID NO: 57 - protein, 17 aa, Mus sp.
EILPSTGRTNYNENFKG

SEQ ID NO: 58 - protein, 17 aa, Mus sp.
RVNPSNGDTKYNQNFKG

SEQ ID NO: 59 - protein, 17 aa, Mus sp.
RIDPANGNTKYAPKFQD

SEQ ID NO: 60 - protein, 16 aa, Mus sp.
VIWAGGPTNYNSALMS

SEQ ID NO: 61 - protein, 16 aa, Mus sp.
VIWTGITTNYNSALIS

SEQ ID NO: 62 - protein, 15 aa, Mus sp.
EINPGNVRTNYNENF

SEQ ID NO: 63 - protein, 16 aa, Mus sp.
VIWSDGSTDFNAPFKS

SEQ ID NO: 64 - protein, 16 aa, Mus sp.
VIWSDGSTDYNAPFKS

SEQ ID NO: 65 - protein, 16 aa, Mus sp.
VIWPVGSTNYNSALMS

SEQ ID NO: 66 - protein, 17 aa, Mus sp.
RIDPANGNTKYDPRFQD

SEQ ID NO: 67 - protein, 10 aa, Mus sp.
VYFGNPWFAY

SEQ ID NO: 68 - protein, 16 aa, Mus sp.
TKNFYSSYSYDDAMDY

SEQ ID NO: 69 - protein, 15 aa, Mus sp.
SFPNNYYSYDDAFAY

SEQ ID NO: 70 - protein, 10 aa, Mus sp.
QIYYSTLVDY

SEQ ID NO: 71 - protein, 12 aa, Mus sp.
GTGTGFYYAMDY

SEQ ID NO: 72 - protein, 10 aa, Mus sp.
VFYGEPYFPY

SEQ ID NO: 73 - protein, 11 aa, Mus sp.
KGGYSGSWFAY

SEQ ID NO: 74 - protein, 9 aa, Mus sp.
MDWDDFFDY

SEQ ID NO: 75 - protein, 15 aa, Mus sp.
SFPDNYYSYDDAFAY

SEQ ID NO: 76 - protein, 108 aa, Artificial Sequence
  Description of Artificial Sequence Synthetic polypeptide
EIVLTQSPGTLSLSPGERATMSCTASSSVSSSYFHWYQQKPGQAPRLLIYRTSTLASGIPDRFSGSGSGTDFTLTISRLEPEDAATYYCHQFHRSPLTFGQGTKLEIK

SEQ ID NO: 77 - protein, 108 aa, Artificial Sequence
  Description of Artificial Sequence Synthetic polypeptide
EIVLTQSPGTLSLSPGERATMSCTASSSVSSSYFHWYQQKPGQAPRLLIYRTSILASGVPDRFSGSGSGTDFTLTISRLEPEDFATYYCHQFHRSPLTFGQGTKLEIK

SEQ ID NO: 78 - protein, 108 aa, Artificial Sequence
  Description of Artificial Sequence Synthetic polypeptide
EIVLTQSPGTLSLSPGERATMSCTASSSVSSSYFHWYQQKPGQAPRLWIYRTSRLASGVPDRFSGSGSGTDFTLTISRLEPEDAATYYCHQFHRSPLTFGQGTKLEIK

SEQ ID NO: 79 - protein, 108 aa, Artificial Sequence
  Description of Artificial Sequence Synthetic polypeptide
EIVLTQSPGTLSLSPGERATMTCTASSSVSSSYFHWYQQKPGQAPRLLIYRTSRLASGVPDRFSGSGSGTDFTLTISRLEPEDFAVYYCHQFHRSPLTFGQGTKLEIK

SEQ ID NO: 80 - protein, 108 aa, Artificial Sequence [sequence omitted]

SEQ ID NO: 81 - protein, 108 aa, Artificial Sequence
  Description of Artificial Sequence Synthetic polypeptide
QIVLTQSPGTLSLSPGERVTMSCTASSSVSSSYFHWYQQKPGQAPRLLIYRTSQLASGIPDRFSGSGSGTDFTLTISRLEPEDAATYYCHQFHRSPLTFGQGTKLEIK

SEQ ID NO: 82 - protein, 108 aa, Artificial Sequence
  Description of Artificial Sequence Synthetic polypeptide
QIVLTQSPGTLSLSPGERATMTCTASSSVSSSYFHWYQQKPGQAPRLLIYRTSKLASGVPDRFSGSGSGTDFTLTISRLEPEDFATYYCHQFHRSPLTFGQGTKLEIK

SEQ ID NO: 83 - protein, 108 aa, Artificial Sequence
  Description of Artificial Sequence Synthetic polypeptide
EIVLTQSPGTLSLSPGERATMSCTASSSVSSSYFHWYQQKPGQAPRLLIYRTSHLASGIPGRFSGSGSGTDFTLTISRLEPEDAAVYYCHQFHRSPLTFGQGTKLEIK

SEQ ID NO: 84 - protein, 107 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPDRFSGSGSGTEFTLTISSLQSEDFAEYFCQQYSRYPLTFGQGTKLEIK

SEQ ID NO: 85 - protein, 107 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPDRFSGSGSGTEFTLTISSLQSEDFAVYYCQQYSRYPLTFGQGTKLEIK

SEQ ID NO: 86 - protein, 107 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPARFSGSGSGTEFTLTISSLQSEDFAEYYCQQYSRYPLTFGQGTKLEIK

SEQ ID NO: 87 - protein, 119 aa, Artificial Sequence
  Description of Artificial Sequence Synthetic polypeptide
QVQLVQSGAEVKKPGASVKVSCKASGYSFTSSWIHWVRQAPGQGLEWIGEINPGNVRTNYNENFRNKATMTVDTSISTAYMELSRLRSDDTAVYYCAVVFYGEPYFPYWGQGTLVTVSS

SEQ ID NO: 88 - protein, 119 aa, Artificial Sequence
  Description of Artificial Sequence Synthetic polypeptide
QVQLVQSGAEVKKPGASVKVSCKASGYSFTSSWIHWVRQRPGQGLEWIGEINPGNVRTNYNENFRNRVTMTVDTSISTAYMELSRLRSDDTAVYYCTVVFYGEPYFPYWGQGTLVTVSS

SEQ ID NO: 89 - protein, 119 aa, Artificial Sequence [sequence omitted]

SEQ ID NO: 90 - protein, 119 aa, Artificial Sequence
  Description of Artificial Sequence Synthetic polypeptide
QVQLVQSGAEVKKPGASVKVSCKASGYSFTSSWIHWVRQRPGQGLEWMGEINPGNVRTNYNENFRNRATLTRDTSISTAYMELSRLRSDDTAVYYCAVVFYGEPYFPYWGQGTLVTVSS

SEQ ID NO: 91 - protein, 119 aa, Artificial Sequence
  Description of Artificial Sequence Synthetic polypeptide
QVQLVQSGAEVKKPGASVKVSCKASGYSFTSSWIHWVRQRPGQGLEWMGEILPGVVRTNYNENFRNKVTMTVDTSISTAYMELSRLRSDDTAVYYCTVVFYGEPYFPYWGQGTLVTVSS

SEQ ID NO: 92 - protein, 119 aa, Artificial Sequence
  Description of Artificial Sequence Synthetic polypeptide
QVQLVQSGAEVKKPGASVKVSCKASGYSFTSSWIHWVRQRPGQGLEWIGEINPGAVRTNYNENFRNRVTMTVDTSISTAYMELSRLRSDDTAVYYCTVVFYGEPYFPYWGQGTLVTVSS

SEQ ID NO: 93 - protein, 119 aa, Artificial Sequence
  Description of Artificial Sequence Synthetic polypeptide
QVQLVQSGAEVKKPGASVKVSCKASGYSFTSSWIHWVRQAPGQGLEWIGEINPGLVRTNYNENFRNKVTMTVDTSISTAYMELSRLRSDDTAVYYCAVVFYGEPYFPYWGQGTLVTVSS

SEQ ID NO: 94 - protein, 119 aa, Artificial Sequence
  Description of Artificial Sequence Synthetic polypeptide
QVQLVQSGAEVKKPGASVKVSCKASGYSFTSSWIHWVRQAPGQGLEWIGEINPGAVRTNYNENFRNKVTMTVDTSISTAYMELSRLRSDDTAVYYCAVVFYGEPYFPYWGQGTLVTVSS

SEQ ID NO: 95 - protein, 119 aa, Artificial Sequence
  Description of Artificial Sequence Synthetic polypeptide
QVQLVQSGAEVKKPGASVKVSCKASGYSFTSSWIHWVRQAPGQGLEWIGEINPGSVRTNYNENFRNKATMTVDTSISTAYMELSRLRSDDTAVYYCAVVFYGEPYFPYWGQGTLVTVSS

SEQ ID NO: 96 - protein, 119 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTINKDTSKSQVSFKMSSVQAADTAVYYCARKGGYSGSWFAYWGQGTLVTVSS

SEQ ID NO: 97 - protein, 119 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDTSKNQVSLKMNSLTTDDTAVYYCARKGGYSGSWFAYWGQGTLVTVSS

SEQ ID NO: 98 - protein, 119 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDNSKSQVSLKMNSVTVADTAVYYCARKGGYSGSWFAYWGQGTLVTVSS

SEQ ID NO: 99 - protein, 119 aa, Artificial Sequence
  Description of Artificial Sequence Synthetic polypeptide
QVQLQESGPGLVKPSETLSITCTVSGFSLTDYAVHWVRQPPGKGLEWIGVIWSDGSTDYNAPFKSRVTISKDTSKNQVSFKLSSVTVDDTAVYYCARKGGYSGSWFAYWGQGTLVTVSS

SEQ ID NO: 100 - protein, 119 aa, Artificial Sequence
  Description of Artificial Sequence Synthetic polypeptide
QVQLQESGPGLVAPSETLSLTCTVSGFSLTDYAVHWIRQFPGKGLEWIGVIWSDGSTDFNAPFKSRVTISKDTSKNQVSFKLSSVTTDDTAVYYCARKGGYSGSWFAYWGQGTLVTVSS

SEQ ID NO: 101 - protein, 119 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDNSKSQVSFKMSSVTADDTAVYYCARKGGYSGSWFAYWGQGTLVTVSS

SEQ ID NO: 102 - protein, 7 aa, Mus sp.
RTSTLAS

SEQ ID NO: 103 - protein, 7 aa, Mus sp.
RTSILAS

SEQ ID NO: 104 - protein, 7 aa, Mus sp.
RTSRLAS

SEQ ID NO: 105 - protein, 7 aa, Mus sp.
RTSQLAS

SEQ ID NO: 106 - protein, 7 aa, Mus sp.
RTSKLAS

SEQ ID NO: 107 - protein, 10 aa, Mus sp.
GFSLTDYAVH

SEQ ID NO: 108 - protein, 15 aa, Mus sp.
EILPGVVRTNYNENF

SEQ ID NO: 109 - protein, 15 aa, Mus sp.
EINPGAVRTNYNENF

SEQ ID NO: 110 - protein, 15 aa, Mus sp.
EINPGLVRTNYNENF

SEQ ID NO: 111 - protein, 15 aa, Mus sp.
EINPGSVRTNYNENF

SEQ ID NO: 112 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 107 aa, Artificial Sequence [sequence omitted]

SEQ ID NO: 114 - protein, 215 aa, Artificial Sequence
  Description of Artificial Sequence Synthetic polypeptide
EIVLTQSPGTLSLSPGERATMSCTASSSVSSSYFHWYQQKPGQAPRLLIYRTSTLASGIPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 215 aa, Artificial Sequence
  Description of Artificial Sequence Synthetic polypeptide
EIVLTQSPGTLSLSPGERATMSCTASSSVSSSYFHWYQQKPGQAPRLLIYRTSILASGVPDRFSGSGSGTDFTLTISRLEPEDFATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 215 aa, Artificial Sequence
  Description of Artificial Sequence Synthetic polypeptide
EIVLTQSPGTLSLSPGERATMSCTASSSVSSSYFHWYQQKPGQAPRLWIYRTSRLASGVPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 215 aa, Artificial Sequence
  Description of Artificial Sequence Synthetic polypeptide
EIVLTQSPGTLSLSPGERATMTCTASSSVSSSYFHWYQQKPGQAPRLLIYRTSRLASGVPDRFSGSGSGTDFTLTISRLEPEDFAVYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 215 aa, Artificial Sequence
  Description of Artificial Sequence Synthetic polypeptide
QIVLTQSPGTLSLSPGERATMTCTASSSVSSSYFHWYQQKPGQAPRLWIYRTSRLASGVPDRFSGSGSGTDFTLTISRLEPEDAATYYCHQFHRSPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 215 aa, Artificial Sequence
  Description of Artificial Sequence Synthetic polypeptide
QIVLTQSPGTLSLSPGERVTMSCTASSSVSSSYFHWYQQKPGQAPRLLIYRTSQLASGIPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 215 aa, Artificial Sequence
  Description of Artificial Sequence Synthetic polypeptide
QIVLTQSPGTLSLSPGERATMTCTASSSVSSSYFHWYQQKPGQAPRLLIYRTSKLASGVPDRFSGSGSGTDFTLTISRLEPEDFATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 215 aa, Artificial Sequence
  Description of Artificial Sequence Synthetic polypeptide
EIVLTQSPGTLSLSPGERATMSCTASSSVSSSYFHWYQQKPGQAPRLLIYRTSHLASGIPGRFSGSGSGTDFTLTISRLEPEDAAVYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 214 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPDRFSGSGSGTEFTLTISSLQSEDFAEYFCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 214 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPDRFSGSGSGTEFTLTISSLQSEDFAVYYCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 214 aa, Artificial Sequence
  Description of Artificial Sequence Synthetic polypeptide
EIVMTQSPATLSVSPGVRATLSCKASQDVGTNVLWYQQKPGQAPRPLIYSASYRHSGIPARFSGSGSGTEFTLTISSLQSEDFAEYYCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 449 aa, Artificial Sequence
  Description of Artificial Sequence Synthetic polypeptide
QVQLVQSGAEVKKPGASVKVSCKASGYSFTSSWIHWVRQAPGQGLEWIGEINPGNVRTNYNENFRNKATMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 449 aa, Artificial Sequence
  Description of Artificial Sequence Synthetic polypeptide
QVQLVQSGAEVKKPGASVKVSCKASGYSFTSSWIHWVRQRPGQGLEWIGEINPGNVRTNYNENFRNRVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 449 aa, Artificial Sequence
  Description of Artificial Sequence Synthetic polypeptide
QVQLVQSGAEVKKPGASVKVSCKASGYSFTSSWIHWVKQAPGQGLEWMGEINPGNVRTNYNENFRNKVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 449 aa, Artificial Sequence
  Description of Artificial Sequence Synthetic polypeptide
QVQLVQSGAEVKKPGASVKVSCKASGYSFTSSWIHWVRQRPGQGLEWMGEINPGNVRTNYNENFRNRATLTRDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 449 aa, Artificial Sequence
  Description of Artificial Sequence Synthetic polypeptide
QVQLVQSGAEVKKPGASVKVSCKASGYSFTSSWIHWVRQRPGQGLEWMGEILPGVVRTNYNENFRNKVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 449 aa, Artificial Sequence
  Description of Artificial Sequence Synthetic polypeptide
QVQLVQSGAEVKKPGASVKVSCKASGYSFTSSWIHWVRQRPGQGLEWIGEINPGAVRTNYNENFRNRVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 449 aa, Artificial Sequence
  Description of Artificial Sequence Synthetic polypeptide
QVQLVQSGAEVKKPGASVKVSCKASGYSFTSSWIHWVRQAPGQGLEWIGEINPGLVRTNYNENFRNKVTMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 449 aa, Artificial Sequence
  Description of Artificial Sequence Synthetic polypeptide
QVQLVQSGAEVKKPGASVKVSCKASGYSFTSSWIHWVRQAPGQGLEWIGEINPGAVRTNYNENFRNKVTMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 449 aa, Artificial Sequence
  Description of Artificial Sequence Synthetic polypeptide
QVQLVQSGAEVKKPGASVKVSCKASGYSFTSSWIHWVRQAPGQGLEWIGEINPGSVRTNYNENFRNKATMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 449 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTINKDTSKSQVSFKMSSVQAADTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 449 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDTSKNQVSLKMNSLTTDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 449 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDNSKSQVSLKMNSVTVADTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 449 aa, Artificial Sequence
  Description of Artificial Sequence Synthetic polypeptide
QVQLQESGPGLVKPSETLSITCTVSGFSLTDYAVHWVRQPPGKGLEWIGVIWSDGSTDYNAPFKSRVTISKDTSKNQVSFKLSSVTVDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 449 aa, Artificial Sequence
  Description of Artificial Sequence Synthetic polypeptide
QVQLQESGPGLVAPSETLSLTCTVSGFSLTDYAVHWIRQFPGKGLEWIGVIWSDGSTDFNAPFKSRVTISKDTSKNQVSFKLSSVTTDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 449 aa, Artificial Sequence
  Description of Artificial Sequence Synthetic polypeptide
QVQLQESGPGLVKPSETLSITCTVSGFSLTDYAVHWIRQPPGKGLEWIGVIWSDGSTDYNAPFKSRVTISKDNSKSQVSFKMSSVTADDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 7 aa, Mus sp.
RTSHLAS

SEQ ID NO: 141 - protein, 5 aa, Mus sp.
SSWIH

SEQ ID NO: 142 - protein, 17 aa, Mus sp.
EINPGNVRTNYNENFRN